(19) United States
(12) Patent Application Publication
(10) Pub. No.: US 2021/0262016 A1
(43) Pub. Date: Aug. 26, 2021

(54) METHODS AND SYSTEMS FOR SOMATIC MUTATIONS AND USES THEREOF

(71) Applicant: MYRIAD GENETICS, INC., Salt Lake City, UT (US)

(72) Inventors: Andrey ZHARKIKH, Salt Lake City, UT (US); Kirsten TIMMS, Salt Lake City, UT (US); Michael PERRY, Salt Lake City, UT (US); Alexander GUTIN, Salt Lake City, UT (US)

(73) Assignee: MYRIAD GENETICS, INC., Salt Lake City, UT (US)

(21) Appl. No.: 17/313,946

(22) Filed: May 6, 2021

Related U.S. Application Data
(63) Continuation of application No. PCT/US19/61036, filed on Nov. 12, 2019.
(60) Provisional application No. 62/760,743, filed on Nov. 13, 2018.
(60) Provisional application No. 62/929,554, filed on Nov. 1, 2019.

(51) Int. Cl. C12Q 1/6827; C12Q 1/6869; C12Q 1/6886; G16B 20/20; G16B 30/20

(52) U.S. Cl. CPC C12Q 1/6827; C12Q 1/6869; C12Q 1/6886; C12Q 2600/118; G16B 30/20; C12Q 2600/156; C12Q 2600/106; G16B 20/20

(57) ABSTRACT

This invention provides methods and compositions for detecting somatic mutations in cancer cells. The methods can be used for measuring tumor mutation burden. Provided are methods for identifying and treating subjects who benefit from treatment with anticancer agents such as immune checkpoint inhibitors, methods for treating cancer in a subject, and methods for monitoring and prognosing a subject having cancer.

### CROSS-REFERENCE TO RELATED APPLICATIONS

This application is a continuation of International Application No. PCT/US2019/061036, filed Nov. 12, 2019, which claims the benefit of priority to U.S. Provisional Application No. 62/760,743, filed Nov. 13, 2018, and to U.S. Provisional Application No. 62/929,554, filed Nov. 1, 2019, the contents of each of which are hereby incorporated by reference.

### TECHNICAL FIELD

This invention relates to methods, compositions, kits and systems for detecting somatic mutations in cancer cells by nucleic acid sequencing. More particularly, this disclosure provides methods for measuring a tumor mutation burden, for identifying and treating subjects who benefit from treatment with anticancer agents, such as immune checkpoint inhibitors, as well as for treating cancer in a subject, and for monitoring and prognosing a subject having cancer

### BACKGROUND

One of the hallmarks of cancer in cells is the presence of somatic variants in the genome. See, e.g. Theodor Boveri, J. Cell Sci. (2008) 121:1-84. Somatic variants can be used as a biomarkers for cancer, particularly when the frequency of variants can be accurately detected and recorded. However, it is difficult to detect somatic variants quantitatively.

The frequency of somatic variants in cancer cells can range from below 0.1 up to several hundred per Mb. Drawbacks of methods for detecting somatic variants include low sensitivity because of the low frequencies of appearance of the variants. Attempts to identify and count somatic variants at low frequencies may not overcome the level of noise in high throughput nucleic acid sequencing methodologies.

Further, in nucleic acid sequencing methodologies that require a reference genome, insufficient representation of various alleles in the reference genome can lead to inaccuracies due to group or ethnic bias.

A significant drawback in some conventional sequencing methodologies is the need for a non-cancer germline comparator sample to be used to distinguish germline variants from the variants detected in cancer samples. The non-cancer germline comparator sample can provide a baseline to be subtracted from the somatic variants detected in cancer cells. In fact, in many cases such comparator samples may not even be available.

What is needed are methods, compositions and systems for detecting somatic variants with high sensitivity. It is also desirable to improve sequencing methodologies to accurately detect and count somatic variants.

There is an urgent need for methods for treating cancer and to identify subjects who benefit from treatment. What is needed are methods and systems that do not require a non-cancer comparator sample along with the sample of a tumor or tissue from a subject having cancer.

There has long been a need to achieve these goals by methods involving direct detection of variants to reduce errors.

### BRIEF SUMMARY

This invention provides methods, compositions, kits and systems for detecting somatic mutations in cancer cells, for identifying and treating subjects who benefit from treatment with anticancer agents such as immune checkpoint inhibitors, for measuring a tumor mutation burden, for treating cancer in a subject, and for monitoring and prognosing a subject having cancer.

The measurement of somatic mutations can provide therapeutic, diagnostic, and prognostic methods for cancer.

In some aspects, this invention provides methods for selecting and identifying subjects who benefit from a treatment, such as a treatment for cancer using an anticancer agent. For such subjects, a therapeutic modality can be selected for treating cancer.

In further aspects, this invention provides methods for measuring and scoring tumor mutation frequency in cancer cells. The scores can be used to calculate a tumor mutation burden for a sample from a subject. The tumor mutation burden can serve as a biomarker for a disease such as cancer.

Somatic variants may be associated with the response of a subject to treatment using certain medicaments. For example, high tumor mutation burden values may be associated with favorable response of a subject having cancer to administration of an immune checkpoint inhibitor drug.

Embodiments of this Invention Include:

A method for detecting a somatic variant, comprising:

(a) sequencing cells of a sample;
(b) identifying a set of heterozygous SNP positions, wherein each SNP has alleles B and A;
(c) detecting two germline allele parings for a SNP position and a variant in a position near the SNP position, wherein the two germline allele parings are (i) allele B and a first variant allele, and (ii) allele A and a second variant allele which may the same or different than the first variant allele; and
(d) detecting a third allele pairing which is (iii) allele B and a third variant allele that is different from the first variant allele. The allele pairings can each be detected in a contiguous nucleic acid sequence containing one of the SNP positions, so that the variant position is within one detection length of the SNP position. The contiguous nucleic acid sequence can be a read length of about 100 to 5000 bases. The detection length may be 200 to 1000 contiguous base positions on each flank of the SNP position. The method does not utilize a separate germline comparator sample. The sample can be a cancer tissue sample, a sample of tumor cells, or a tumor sample. The amount of non-tumor cells in the sample may be minimized. The sample may contain non-tumor cells. The allele pairings can be detected by massively parallel sequencing, by hybridization, or with amplification. The set of heterozygous SNP positions may be at least 500 SNP positions, or at least 1000 SNP positions, or at least 5000 SNP positions. The method can detect a somatic variant at a minimum level of 0.1 per Mb, or 0.3 per Mb, or 0.7 per Mb. The detecting may be obtained with a targeted SNP panel. The detecting can be obtained by fragmentation sequencing that uses a human reference genome.

A method for detecting a somatic variant, comprising:

(a) sequencing cells of a tumor sample;

(b) obtaining sequence reads from the sample using a massively parallel nucleic acid sequencing process, wherein the sequence reads have a read length;

(c) mapping the sequence reads to a reference genome;

(d) assembling a somatic variant count matrix of sequence reads that are mapped to a heterozygous-SNP position of the reference genome, wherein the count matrix has first and second elements which count allele pairings of SNP alleles B and A, respectively, to a variant allele, and wherein the count matrix has a third element which counts read sequences from SNP allele B paired to a different variant allele than in the first element; and

(e) calculating a somatic mutation significance score (S) for the third element. The method does not utilize a separate germline comparator sample. The sample may be a cancer tissue sample, a sample of tumor cells, or a tumor sample. The method can detect a somatic variant at a minimum level of 0.1 per Mb, or 0.3 per Mb, or 0.7 per Mb. The sequence reads may be obtained with a targeted SNP panel. The read length may be 100 to 5000, or 200 to 1000 contiguous base positions. The average read depth may be at least 50x or 100x for the portion of the reference genome covered. The reference genome can be a human genome. The sequence reads may be error-filtered and position-filtered. The somatic mutation significance score (S) is given by Formula I

S=(C(Z,P)²/(C(Z,P)+C(X,P))+(C(Z,P)−E)²/E)/2*10 Formula I

wherein C(Z,P) is the third element count, C(X,P) is the first element count, and E is an error rate calculated from the average of all other counts in the matrix, except for the highest three counts, for all SNP regions.

A method for identifying a subject having cancer who benefits from a treatment, the method comprising:

(a) sequencing cells of a tumor sample from the subject;
(b) identifying a set of heterozygous SNP positions, wherein each SNP has alleles B and A;
(c) detecting two germline allele parings for a SNP position and a variant in a position near the SNP position, wherein the two germline allele parings are (i) allele B and a first variant allele, and (ii) allele A and a second variant allele which may the same or different than the first variant allele; and
(d) detecting a third allele pairing which is (iii) allele B and a third variant allele that is different from the first variant allele, wherein the third allele pairing arises from a somatic variant;
(f) calculating a value for a tumor mutation burden from the somatic variants detected from the allele pairings; and
(g) identifying the subject having cancer who benefits from a treatment who has the tumor mutation burden greater than a reference level.

A method for identifying a subject having cancer who benefits from a treatment, the method comprising:

(a) sequencing cells of a tumor sample from the subject;

(b) obtaining sequence reads from the sample using a massively parallel nucleic acid sequencing process, wherein the sequence reads have a read length;

(c) mapping the sequence reads to a reference genome;

(d) assembling a somatic variant count matrix of sequence reads that are mapped to a heterozygous-SNP position of the reference genome, wherein the count matrix has first and second elements which count allele pairings of SNP alleles B and A, respectively, to a variant allele, and wherein the count matrix has a third element which counts read sequences from SNP allele B paired to a different variant allele than in the first element;

(e) calculating a value for a tumor mutation burden of the sample by the steps:
(i) calculating a somatic mutation significance score (S) for the third element; and
(ii) calculating the value for the tumor mutation burden from the number of somatic variants having a somatic mutation significance score above a threshold, normalized by the total number of positions in the heterozygous-SNP regions; and

(f) identifying the subject having cancer who benefits from a treatment who has the tumor mutation burden greater than a reference level of somatic mutation. The number of heterozygous-SNPs in the reference genome may be from about 100 up to the total number of heterozygous-SNPs in the reference genome. The reference level of somatic mutation may be a level for which the subject will benefit from the treatment. The reference level of somatic mutation can be the average tumor mutation burden of the reference genome. The reference level of somatic mutation may be the average tumor mutation burden of a reference population having the same kind of cancer as the subject. The reference level of somatic mutation can be the average tumor mutation burden of a reference population not having cancer. The reference level of somatic mutation may be the average tumor mutation burden of a reference population that does not benefit from the treatment. The reference level of somatic mutation can be obtained with a different sample from the subject. The tumor mutation burden threshold may be 15, or 20, or 30, or 40, and the tumor mutation burden is given by Formula II

TMB=N(S>threshold)/(N(HomHet)+N(HetHet))*1000000 Formula II

wherein N is the number of somatic variants having a somatic mutation significance score above the threshold, normalized by the total number of positions in the heterozygous-SNP regions (N(HomHet)+N(HetHet)).

A method for treating cancer in a subject in need thereof, the method comprising:

(a) sequencing cells of a tumor sample from the subject;
(b) identifying a set of heterozygous SNP positions, wherein each SNP has alleles B and A;
(c) detecting two germline allele parings for a SNP position and a variant in a position near the SNP position, wherein the two germline allele parings are (i) allele B and a first variant allele, and (ii) allele A and a second variant allele which may the same or different than the first variant allele; and
(d) detecting a third allele pairing which is (iii) allele B and a third variant allele that is different from the first variant allele, wherein the third allele pairing arises from a somatic variant;
(e) calculating a value for a tumor mutation burden from the somatic variants detected;
(f) identifying the subject having cancer who benefits from a treatment who has the tumor mutation burden greater than a reference level; and
(g) administering a treatment for cancer.

A method for treating cancer in a subject in need thereof, the method comprising:

(a) sequencing cells of a tumor sample from the subject;

(b) obtaining sequence reads from the sample using a massively parallel nucleic acid sequencing process, wherein the sequence reads have a read length;

(c) mapping the sequence reads to a reference genome;

(d) assembling a somatic variant count matrix of sequence reads that are mapped to a heterozygous-SNP position of the reference genome, wherein the count matrix has first and second elements which count allele pairings of SNP alleles B and A, respectively, to a variant allele, and wherein the count matrix has a third element which counts read sequences from SNP allele B paired to a different variant allele than in the first element;

(e) calculating a value for a tumor mutation burden of the sample by the steps:
(i) calculating a somatic mutation significance score (S) for the third element for each somatic variant; and
(ii) calculating the value for the tumor mutation burden from the number of somatic variants having a somatic mutation significance score above a threshold, normalized by the total number of positions in the heterozygous-SNP regions;

(f) identifying the subject having cancer who will benefit from a treatment who has the tumor mutation burden greater than a reference level of somatic mutation; and

(g) administering a treatment for cancer. The treatment for cancer may comprise administering an immune checkpoint inhibitor drug.

A method for treating cancer in a subject in need thereof, the method comprising:

(a) sequencing cells of a tumor sample from the subject;

(b) obtaining sequence reads from the sample using a massively parallel nucleic acid sequencing process, wherein the sequence reads have a read length;

(c) mapping the sequence reads to a reference genome;

(d) assembling a somatic variant count matrix of sequence reads that are mapped to a heterozygous-SNP position of the reference genome, wherein the count matrix has first and second elements which count allele pairings of SNP alleles B and A, respectively, to a variant allele, and wherein the count matrix has a third element which counts read sequences from SNP allele B paired to a different variant allele than in the first element;

(e) calculating a value for a tumor mutation burden of the sample by the steps:
(i) calculating a somatic mutation significance score (S) for the third element for each somatic variant; and
(ii) calculating the value for the tumor mutation burden from the number of somatic variants having a somatic mutation significance score above a threshold, normalized by the total number of positions in the heterozygous-SNP regions;

(f) identifying a subject having cancer who will benefit from a treatment who has the tumor mutation burden greater than a reference level of somatic mutation;

(g) monitoring the subject for the signs and symptoms of cancer for a period of time; and

(h) administering a treatment for cancer. The treatment may be administering an immune checkpoint inhibitor.

A method for monitoring a response of a subject having cancer to a treatment, the method comprising:

(a) sequencing cells of a tumor sample from the subject;
(b) identifying a set of heterozygous SNP positions, wherein each SNP has alleles B and A;
(c) detecting two germline allele parings for a SNP position and a variant in a position near the SNP position, wherein the two germline allele parings are (i) allele B and a first variant allele, and (ii) allele A and a second variant allele which may the same or different than the first variant allele; and
(d) detecting a third allele pairing which is (iii) allele B and a third variant allele that is different from the first variant allele, wherein the third allele pairing arises from a somatic variant;
(e) calculating a value for a tumor mutation burden from the somatic variants detected.

A method for monitoring a response of a subject having cancer to a treatment, the method comprising:

(a) sequencing cells of a tumor sample from the subject;

(b) obtaining sequence reads from the sample using a massively parallel nucleic acid sequencing process, wherein the sequence reads have a read length;

(c) mapping the sequence reads to a reference genome;

(d) assembling a somatic variant count matrix of sequence reads that are mapped to a heterozygous-SNP position of the reference genome, wherein the count matrix has first and second elements which count allele pairings of SNP alleles B and A, respectively, to a variant allele, and wherein the count matrix has a third element which counts read sequences from SNP allele B paired to a different variant allele than in the first element;

(e) calculating a value for a tumor mutation burden of the sample by the steps:
(i) calculating a somatic mutation significance score (S) for the third element for each somatic variant; and
(ii) calculating the value for the tumor mutation burden from the number of somatic variants having a somatic mutation significance score above a threshold, normalized by the total number of positions in the heterozygous-SNP regions.

A method for prognosing a subject having cancer, the method comprising:

(a) sequencing cells of a tumor sample from the subject;
(b) identifying a set of heterozygous SNP positions, wherein each SNP has alleles B and A;
(c) detecting two germline allele parings for a SNP position and a variant in a position near the SNP position, wherein the two germline allele parings are (i) allele B and a first variant allele, and (ii) allele A and a second variant allele which may the same or different than the first variant allele; and
(d) detecting a third allele pairing which is (iii) allele B and a third variant allele that is different from the first variant allele, wherein the third allele pairing arises from a somatic variant;
(e) calculating a value for a tumor mutation burden from the somatic variants detected; and
(f) prognosing the subject as having a poor prognosis who has the tumor mutation burden greater than a TMB reference level.

A method for prognosing a subject having cancer, the method comprising:

(a) sequencing cells of a tumor sample from the subject;

(b) obtaining sequence reads from the sample using a massively parallel nucleic acid sequencing process, wherein the sequence reads have a read length;

(c) mapping the sequence reads to a reference genome;

(d) assembling a somatic variant count matrix of sequence reads that are mapped to a heterozygous-SNP position of the reference genome, wherein the count matrix has first and second elements which count allele pairings of SNP alleles B and A, respectively, to a variant allele, and wherein the count matrix has a third element which counts read sequences from SNP allele B paired to a different variant allele than in the first element;

(e) calculating a value for a tumor mutation burden of the sample by the steps:
(i) calculating a somatic mutation significance score (S) for the third element for each somatic variant; and
(ii) calculating the value for the tumor mutation burden from the number of somatic variants having a somatic mutation significance score above a threshold, normalized by the total number of positions in the heterozygous-SNP regions;

(f) prognosing the subject as having a poor prognosis who has the tumor mutation burden greater than a TMB reference level; and

(g) administering a treatment for cancer.

A kit for identifying a subject having cancer who benefits from a treatment, the kit comprising:

(a) reagents for obtaining sequence reads from a sample from the subject, wherein the sequence reads can be used to obtain a value for a tumor mutation burden of the sample; and

(b) instructions for using the reagents for obtaining the sequence reads and the value for a tumor mutation burden for identifying the subject.

A system for detecting a somatic variant, comprising:

means for receiving, enriching and amplifying a nucleic acid from a sample, wherein the sample contains cancer cells and non-cancer cells;

means for synthesizing a library from the nucleic acid;

means for contacting the library with a sequencing chip;

means for detecting a sequence in the library and transferring sequence data to a processor;

one or more processors for carrying out the steps:
(a) providing a sample which contains cancer cells and non-cancer cells;
(b) obtaining sequence reads from the sample using a massively parallel nucleic acid sequencing process, wherein the sequence reads have a read length;
(c) mapping the sequence reads to a reference genome;
(d) assembling a somatic variant count matrix of sequence reads that are mapped to a heterozygous-SNP position of the reference genome, wherein the count matrix has first and second elements which count allele pairings of SNP alleles B and A, respectively, to a variant allele, and wherein the count matrix has a third element which counts read sequences from SNP allele B paired to a different variant allele than in the first element;
(e) calculating a value for a tumor mutation burden of the sample by the steps:
   (i) calculating a somatic mutation significance score (S) for the third element for each somatic variant; and
   (ii) calculating the value for the tumor mutation burden from the number of somatic variants having a somatic mutation significance score above a threshold, normalized by the total number of positions in the heterozygous-SNP regions; and

a display for displaying, charting and reporting sequence information.

A non-transitory machine-readable storage medium having stored therein instructions for execution by a processor which cause the processor to perform the steps of a method for detecting a somatic variant, the method comprising:

(a) providing a sample which contains cancer cells and non-cancer cells;

(b) obtaining sequence reads from the sample using a massively parallel nucleic acid sequencing process, wherein the sequence reads have a read length;

(c) mapping the sequence reads to a reference genome;

(d) assembling a somatic variant count matrix of sequence reads that are mapped to a heterozygous-SNP position of the reference genome, wherein the count matrix has first and second elements which count allele pairings of SNP alleles B and A, respectively, to a variant allele, and wherein the count matrix has a third element which counts read sequences from SNP allele B paired to a different variant allele than in the first element;

(e) calculating a value for a tumor mutation burden of the sample by the steps:
(i) calculating a somatic mutation significance score (S) for the third element for each somatic variant; and
(ii) calculating the value for the tumor mutation burden from the number of somatic variants having a somatic mutation significance score above a threshold, normalized by the total number of positions in the heterozygous-SNP regions; and

(f) displaying, charting and reporting sequence information from the sample.

### BRIEF DESCRIPTION OF THE DRAWINGS

FIG. 1: Illustration of methods and steps for detecting and evaluating tumor mutation burden by nucleic acid sequencing.

FIG. 2. Illustration of germline alleles and germline variants. (top) Germline alleles for a heterozygous variant V/W, which is located near a heterozygous SNP B/A. Each SNP allele is associated with only one variant allele, and only two unique sequence reads are expected, BV and AW, for reads that cover both SNP and VAR positions. (bottom) Germline alleles for a homozygous variant W/W, which is located near a heterozygous SNP B/A. Each SNP allele is associated with only one variant allele, and only two unique sequence reads are expected, BW and AW, for reads that cover both SNP and VAR positions.

FIG. 3. Illustration of somatic alleles and somatic variants. (top) Alleles observed for a heterozygous variant V/W, which is located near a heterozygous SNP B/A. Two unique sequence reads are expected for the two normal allele pairs, BV and AW, for reads that cover both SNP and VAR positions. However, SNP allele B is associated with two variant alleles, BV and BW. Thus, BW represents a de novo mutation. A matrix of these reads shows large (L) counts for BV and AW, and a count (s) for BW, which may be smaller. (bottom) Alleles observed for a homozygous variant W/W, which is located near a heterozygous SNP B/A. Two unique sequence reads are expected for the two normal allele pairs, BW and AW, for reads that cover both SNP and VAR positions. However, SNP allele B is associated with two variant alleles, BV and BW. Thus, BV represents a de novo mutation. A matrix of these reads shows large (L) counts for BW and AW, and a count (s) for BV, which may be smaller.

FIG. 4. Example embodiment of methods for detecting and evaluating tumor mutation burden by nucleic acid sequencing. For a homozygous somatic variant located near a heterozygous SNP (Hom/Het), a sequence read stack was mapped to a reference genome (WT) as shown. A count matrix was assembled which showed the detection of allele pairs GA (count 55), AA (count 32), and AG (count 23). The appearance of the third maximum count AG (count 23) arose from somatic mutations in some cancer cells.

FIG. 5. Example embodiment of methods for detecting and evaluating tumor mutation burden by nucleic acid sequencing. For a heterozygous somatic variant located near a heterozygous SNP (Het/Het), a count matrix was assembled which showed the detection of alleles CG (count 39), GT (count 34), and GG (count 7). The appearance of the third maximum count GG (count 7) arose from somatic mutations in some cancer cells.

FIG. 6. Illustration of sequencing data from colon cancer samples. Each curve represents the number of variant positions (Y axis) by allele ratio % (X axis). One sample showed a large peak representing a high-TMB sample. The tall peak on the left side at very low allele ratio values, less than 10%, reflects sequencing errors which are ignored. For counting the TMB value, the TMB value may be calculated as the area under the curve in the range of allele ratios from about 15% to about 65% for a score greater than 30 (Y axis).

FIG. 7. Plot of data from a SNP-based method of this invention for detecting and evaluating tumor mutation burden in colon and breast cancer samples by nucleic acid sequencing as compared to conventional methods involving subtracting data from a germline comparator sample or germline filtering. Using the direct SNP analysis method of this invention (filled circles) with only a tumor sample, and without a second germline comparator sample, an evaluation of tumor mutation burden was obtained that was surprisingly superior to conventional methods. The sensitivity of the SNP-based method of this invention (filled circles) was surprisingly increased over the conventional methods. More particularly, the SNP-based method of this invention (filled circles) was surprisingly more accurate than a method of nucleic acid sequencing for evaluating tumor mutation burden using a database of known germline variants and filtering of common variants to attempt to remove germline background (open circles).

### DETAILED DESCRIPTION OF THE DISCLOSURE

This invention provides methods, compositions, kits and systems for detecting somatic mutations in cancer cells. The measurement of somatic mutations can provide therapeutic, diagnostic, and prognostic methods for cancer.

In some aspects, this invention provides methods for selecting and identifying subjects who benefit from a treatment, such as a treatment for cancer using an anticancer agent. For such subjects, a therapeutic modality can be selected for treating cancer.

In further aspects, this invention provides methods for measuring and scoring tumor mutation frequency in cancer cells. The scores can be used to calculate a tumor mutation burden for a sample from a subject. The tumor mutation burden can serve as a biomarker for disease, for example, cancer.

Somatic variants may be associated with the response of a subject to treatment using certain medicaments. For example, high tumor mutation burden values may be associated with favorable response of a subject having cancer to administration of an immune checkpoint inhibitor drug.

As used herein, a quantity related to the frequency of somatic variants can be defined as “tumor mutation burden” (TMB). TMB can be calculated as a count of somatic variants in a cancer sample normalized to the total number of genomic positions assayed in determining the count of somatic variants. TMB can be expressed as a number of mutations per megabase of DNA.

TMB can also be measured from RNA and expressed as a number of mutations per megabase of RNA.

A measure of TMB can be obtained as a measure of somatic variants in a set of genomic locations. The set of genomic locations can be a set of SNP regions of the genome.

In some embodiments, a set of heterozygous SNP positions can be identified using sequencing data or sequencing reads.

In some embodiments, a set of heterozygous SNP positions can be identified using known human SNP positions.

A measure of TMB of this invention can be a surrogate for a load of somatic mutations of a genome. A measure of TMB of this invention can provide a numerical level which directly reflects a number of somatic mutations of a genome. A measure of TMB of this invention can provide a numerical level which can be an effective estimate of total mutation load of a genome. A measure of TMB of this invention may differ from a quantity labeled “TMB” in other literature.

In some aspects, this invention provides methods and systems for detecting somatic mutations and determining a mutational level. The mutation load can be obtained from a unique algorithm encompassing detection of somatic mutations in a genome, where the somatic mutations are each located near a SNP position in an array of SNP positions in the genome.

In certain aspects, a measure of TMB of this invention can be obtained from a unique algorithm encompassing detection of a portion of somatic mutations in a genome, where the somatic mutations are each located near a SNP position in an array of SNP positions in the genome.

In further aspects, a measure of TMB of this invention can provide a numerical level which directly reflects a number of somatic mutations of a genome, where a mutation can affect the function of a location in the genome.

In additional aspects, methods of this invention for measuring TMB can utilize data obtained with any sequencing technology which provides multiple independent reads of the locus of interest. In various embodiments, the Sanger sequence method can be utilized.

In further aspects, methods of this invention for measuring TMB can be utilized with any of SNP panels, whole exome/genome sequencing, and gene panels in which SNPs can be sequenced.

In some embodiments, HRD (Myriad Genetics, Inc.) sequencing can be used which is a hybridization capture based gene-panel that also samples SNPs from across the genome. An HRD assay may utilize SNPs to reconstruct a tumor-CN/LOH profile from which an HRD score may be derived. An HRD assay can be used to sequence a large number of SNP loci.

In certain embodiments, any sequencing data with a sufficient number of SNPs, including flanking regions on both sides, can be used.

In further aspects, any sequence based NGS assay may be used in methods of this invention for measuring TMB.

In additional aspects, embodiments of this invention provide methods for treating subjects having cancer. A subject having cancer can be selected and identified by evaluating a tumor mutation burden in a sample from the subject. A subject may be treated with an anticancer agent, such as an effective amount of an immune checkpoint inhibitor.

Aspects of this invention include methods, compositions and systems for detecting somatic variants in a sample with advantageously superior sensitivity, including a measure of TMB of this invention.

This invention can further provide improved methods for sequencing a nucleic acid of a sample. The improved sequencing methodologies of this invention can be used to accurately detect and count somatic variants.

Embodiments described in this disclosure include methods for treating cancer, as well as identifying subjects who benefit from treatment. The unique methods of this invention can be performed with a single sample from a subject, and without a non-cancer comparator sample. Methods of this disclosure provide a direct measure of somatic variants, which can be used to determine a somatic variant score and a value for a tumor mutation burden. The direct measurement of somatic mutations and the evaluation of a tumor mutation burden in a sample from a subject, such as a tumor or tissue sample from a subject having cancer, can provide an accurate biomarker for disease.

Additional aspects of this invention include methods for direct detection of somatic variants, which can reduce errors due to ethnic bias. Methods of this disclosure can detect a somatic variant from a single test sample by counting sequence reads that can be attributed solely to cancer cells. In these methods, a tumor mutation burden can be determined which is pertinent to an individual, and less affected by group or ethnic bias.

A tumor mutation burden determined by methods of this invention can be particularly predictive in certain cancers. The tumor mutation burden can be used to detect and diagnose cancers, as well as determine a prognosis.

Examples of cancers include prostate cancers, melanomas, bladder cancers, breast cancers, hematologic cancers, mesotheliomas, lung cancers, and solid tumors.

In some embodiments, this invention provides methods for evaluating a tumor mutation burden, wherein an abnormal status may indicate a poor prognosis.

In further embodiments, methods for evaluating a tumor mutation burden can be combined with one or more clinical parameters in diagnosing and/or prognosing cancer.

Examples of clinical parameters include, for example, clinical nomograms.

In certain embodiments, a high level of a tumor mutation burden can indicate the presence of a cancer.

In additional embodiments, a high level of a tumor mutation burden can indicate an increased risk of cancer recurrence or progression in a subject for whom a clinical nomogram score indicates a relatively low risk of recurrence or progression.

For example, a high level of a tumor mutation burden can show an increased risk of cancer recurrence or progression independent of tumor grade or stage, or independent of a nomogram score. Thus, a high level of a tumor mutation burden can detect increased risk not detected using clinical parameters alone.

In some aspects, this disclosure provides in vitro diagnostic methods comprising determining at least one clinical parameter for a cancer patient and determining a tumor mutation burden in a sample obtained from the patient.

In some embodiments, abnormal status of a tumor mutation burden can indicate an increased likelihood of recurrence or progression of a cancer.

In certain embodiments, the combination of one or more clinical parameters with evaluation of a tumor mutation burden can improve predictive ability with respect to cancer. In some embodiments more than one clinical parameter may be assessed and combined with evaluation of a tumor mutation burden.

In further aspects, this invention includes in vitro diagnostic methods comprising determining at least one clinical parameter or nomogram score for a patient and evaluating a tumor mutation burden of the patient.

Aspects of this invention include methods for classifying a cancer by evaluating a tumor mutation burden in a tissue or cell sample, more particularly a tumor sample, from a subject.

A tumor sample of this disclosure can contain an admixture of cancer and non-cancer, normal cells. A tumor sample of this disclosure can be obtained so as to minimize the non-cancer or non-tumor content in the sample. For example, the non-tumor content in the sample can be minimized by excising only tumor tissue in a biopsy, or by removing only a lesion with none or minimal normal tissue margin.

In certain embodiments, it is preferable to minimize non-tumor content in the sample so that the measured somatic mutations can be related to a quantity for tumor mutation burden. A tumor mutation burden quantity can be used to characterize the level of de novo or somatic mutations in a tumor.

In additional embodiments, even when a sample contains some non-tumor content, somatic mutations measured can be related to a quantity for tumor mutation burden. A tumor mutation burden quantity can be used to characterize the level of de novo or somatic mutations in a tumor sample for analysis of a clinical state of a subject.

Embodiments of this invention can advantageously utilize samples containing cancer and non-cancer cells in methods for detecting somatic mutations without germline subtraction. Methods of this invention for detecting somatic mutations without germline subtraction can count the number of mutations present only in tumor even in a sample containing an admixture of cancer and non-cancer, normal cells. Methods of this invention for detecting somatic mutations without germline subtraction can identify which mutations are present in normal cells and which are present in tumor cells, and count only the mutations present in tumor.

In some embodiments, a tumor sample of this disclosure can be obtained so as to minimize the non-cancer content in the sample so that somatic mutations can be detected with increased accuracy and/or precision.

In certain embodiments, methods of this invention can advantageously detect somatic mutations in cancer cells without germline subtraction, even in samples containing cancer and non-cancer cells.

A reference value with respect to a tumor mutation burden may represent the average TMB level in a plurality of training patients, for example cancer patients, with similar outcomes whose clinical and follow-up data are available and sufficient to define and categorize the patients by disease outcome, for example recurrence or prognosis.

A reference value for TMB may be a TMB level in a population of subjects having cancer who have been treated with an anticancer agent. In some embodiments, the population may comprise a group of subjects who have been treated with a particular anticancer agent and a different group of subjects that have been treated with a different anticancer agent.

A reference value for TMB may be a TMB level in population of subjects having cancer who do not respond to treatment with an anticancer agent.

In some embodiments, a TMB value can distinguish between subjects who have different responsiveness to treatment with an anticancer agent. In certain embodiments, a TMB value can distinguish subjects who have increased overall survival, or progression-free survival after treatment with an anticancer agent from subjects who do not have increased survival. In additional embodiments, a TMB value can identify subjects of a population who benefit from or respond to a therapeutic treatment.

A “good prognosis value” can be generated from a plurality of training cancer patients characterized as having “good outcome,” for example those who have not had cancer recurrence for a period of time, such as five years, or ten years, or more after initial treatment, or who have not had progression in their cancer five years, or ten years, or more after initial diagnosis.

A “poor prognosis value” can be generated from a plurality of training cancer patients defined as having “poor outcome,” for example those who have had cancer recurrence within five years, or ten years, or more after initial treatment, or who have had progression in their cancer within five years, or ten years, or more after initial diagnosis.

Thus, a good prognosis value may represent an average level of TMB in patients having a “good outcome,” whereas a poor prognosis value may represent an average level of TMB in patients having a “poor outcome.”

In some embodiments, when a value of TMB is increased, a subject may have a poor prognosis.

In certain embodiments, a value of TMB may be increased over a normal value, or a threshold amount.

In various embodiments, a value of TMB may be closer to a poor prognosis value than to a good prognosis value, which can indicate a poor prognosis for the subject.

In other embodiments, a value of TMB may be closer to a good prognosis value than to a poor prognosis value, which can indicate a good prognosis for the subject.

In further embodiments, a TMB value may be determined by assigning patients to risk groups, and a threshold value can be set for the TMB mean.

A threshold value can be selected based on a receiver operating characteristic (ROC) curve, which plots sensitivity versus {1 minus specificity}.

In some embodiments, a TMB reference level can be from about 1 to about 30, or about 2 to about 30, or about 3 to about 30, or about 4 to about 30, or about 5 to about 30, or about 6 to about 30, or about 7 to about 30, or about 8 to about 30, or about 9 to about 30, or about 10 to about 30, or about 10 to about 20 mutations per Mb.

In some embodiments, a TMB reference level can be from about 5 to about 300, or about 10 to about 300, or about 30 to about 300, or about 50 to about 300 mutations per Mb.

In some embodiments, a TMB reference level can be about 1, or about 2, or about 3, or about 4, or about 5, or about 6, or about 7, or about 8, or about 9, or about 10, or about 20 mutations per Mb.

In some embodiments, a TMB reference value can be about 30, or about 50 mutations per Mb.

In general, a cancer may be classified by determining one or more clinically relevant features of the cancer and/or determining a particular prognosis of a patient having the cancer. Thus, “classifying a cancer” may include: (i) evaluating metastatic potential, potential to metastasize to specific organs, risk of recurrence, and/or course of the tumor; (ii) evaluating tumor stage; (iii) determining patient prognosis in the absence of treatment of the cancer; (iv) determining prognosis of patient response (e.g., tumor shrinkage or progression-free survival) to treatment (e.g., chemotherapy, radiation therapy, surgery to excise tumor, etc.); (v) diagnosis of actual patient response to current and/or past treatment; (vi) determining a preferred course of treatment for the patient; (vii) prognosis for patient relapse after treatment (either treatment in general or some particular treatment); (viii) prognosis of patient life expectancy (e.g., prognosis for overall survival).

A “negative classification” refers to an unfavorable clinical feature of a cancer (e.g., a poor prognosis). Examples include (i) an increased metastatic potential, potential to metastasize to specific organs, and/or risk of recurrence; (ii) an advanced tumor stage; (iii) a poor patient prognosis in the absence of treatment of the cancer; (iv) a poor prognosis of patient response (e.g., tumor shrinkage or progression-free survival) to a particular treatment (e.g., chemotherapy, radiation therapy, surgery to excise tumor, etc.); (v) a poor prognosis for patient relapse after treatment (either treatment in general or some particular treatment); (vi) a poor prognosis of patient life expectancy (e.g., prognosis for overall survival).

In some embodiments, a recurrence-associated clinical parameter (or a high nomogram score) and increased TMB may indicate a negative classification in cancer (e.g., increased likelihood of recurrence or progression).

In general, an elevated value of a TMB may accompany rapidly proliferating cancer cells, which may indicate a more aggressive cancer. A subject with an elevated value of a TMB may have an increased likelihood of recurrence after treatment. A subject with an elevated value of a TMB may have an increased likelihood of cancer progression, or more rapid progression, in which rapidly proliferating cells may cause tumors to grow quickly, gain in virulence, and/or metastasize. A subject with an elevated value of a TMB may require a relatively more aggressive treatment.

In some embodiments this invention provides methods for classifying cancer by evaluating a tumor mutation burden, wherein an abnormal status indicates an increased likelihood of recurrence or progression.

In further embodiments, this invention provides methods for determining the prognosis of a cancer in a subject by evaluating a tumor mutation burden, wherein elevated TMB may indicate an increased likelihood of recurrence or progression of the cancer.

In additional embodiments, an assessment can be made before a cancer surgery, for example using a biopsy sample. In other embodiments, an assessment can be made after a cancer surgery, for example using a resected cancer sample.

In certain embodiments, a sample of one or more cells may be obtained from a cancer patient before, during or after treatment.

Examples of cancer treatment include surgical removal of an affected organ, radiotherapy, hormonal therapy (e.g., using GnRH antagonists, GnRH agonists, antiandrogens), chemotherapy, and high intensity focused ultrasound.

Active surveillance of a cancer subject includes observation and regular monitoring without invasive treatment. Active treatment can be started during or after surveillance if symptoms develop, or if there are signs that the cancer growth is progressing or accelerating.

Active surveillance may involve increased risk of cancer metastasis. Surveillance may proceed for one or more months, or one or more years, or longer.

This invention can provide methods for treating a cancer patient or providing guidance for selecting the treatment of a patient. In this method, evaluation of TMB and one or more recurrence-associated clinical parameters may be determined. Active treatment may be recommended, initiated or continued if a sample from the patient has an elevated TMB and the patient has one or more recurrence-associated clinical parameters. Active surveillance may be recommended, or initiated, or continued if the patient has neither an elevated TMB, nor a recurrence-associated clinical parameter. In certain embodiments, TMB, or TMB and one or more clinical parameters may indicate that active treatment is recommended, or that a particular active treatment is recommended, or that aggressive treatment is recommended.

In general, adjuvant therapy (e.g., chemotherapy, radiotherapy, HIFU, hormonal therapy, etc. after prostatectomy or radiotherapy) may be recommended for aggressive disease.

### Methods for Detecting Somatic Mutations

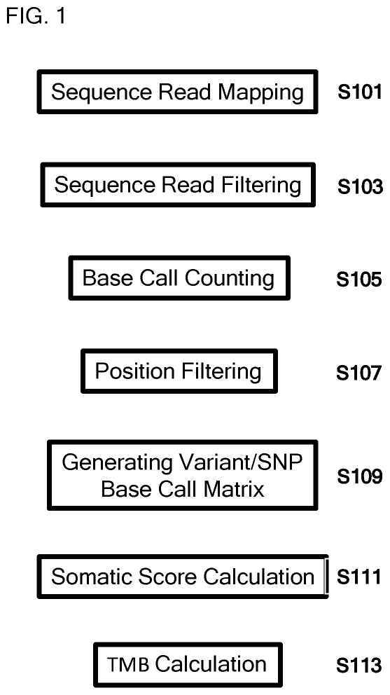

Referring to FIG. 1, this disclosure includes methods for detecting somatic mutations and evaluating a tumor mutation burden of a genome by nucleic acid sequencing.

In a method for detecting a somatic variant, in step S101 sequence reads can be obtained from a sample containing cancer cells and non-cancer cells using a massively parallel nucleic acid sequencing process. The sequence reads can have a read length ranging from about 50 up to about 5000 nucleotides. The sequence reads can be mapped to a reference genome. The sequence reads can be error-filtered in step S103. Base calls of the nucleotides can be counted in step S105, and position filtering can be performed in step S107. A somatic variant-SNP sequence read base call count matrix can be assembled in step S109. The count matrix can use a set of heterozygous-SNP regions of the reference genome. For each heterozygous-SNP position, the count matrix has first and second elements which count only read sequences having at least a first variant located within one read length of the heterozygous-SNP position and a third element which counts only read sequences from a cancer cell having at least a somatic second variant located within one read length of the heterozygous-SNP position. In step S111, a somatic mutation significance score (S) can be calculated for the third element for each somatic variant located within one read length of a heterozygous-SNP position. In step S113, a tumor mutation burden can be calculated for the sample based on the somatic mutation significance scores.

A set of heterozygous-SNP regions can be qualified based on a group of individuals not related to the patient.

In certain embodiments, thorough filtering of the positions can be done to remove polymorphic positions. A position having variants in more than one sample may be considered polymorphic. The presence of related individuals may duplicate the variation and create false polymorphic positions. Thus, before identifying the polymorphism, a set of non-related individuals can be used.

The SNP position set may be predetermined. Positions can be qualified if they are non-repetitive, non-polymorphic and non-prone to a high error rate. This can be estimated from a statistics based on, for example, about 100 or more non-related individuals previously analyzed, or about 50 or more non-related individuals, or about 20 or more non-related individuals, or about 10 or more non-related individuals.

In certain embodiments, the number of qualified positions used for calculating TMB can be 1000 or more, or 5000 or more, or 100,000 or more, or 300,000 or more, or 500,000 or more, or 1,000,000 or more, or 1,500,000 or more, or 1,700,000 or more, or 1,900,000 or more, or 2,000,000 or more.

In some embodiments, the number of qualified positions used for calculating TMB can be at least 1000, or at least 5000, or at least 100,000, or at least 300,000, or at least 500,000, or at least 1,000,000, or at least 1,500,000, or at least 1,700,000, or at least 1,900,000, or at least 2,000,000.

In some embodiments, the number of qualified positions used for calculating TMB can be from 1000 to 3,000,000, or from 5000 to 2,500,000, from 100,000 to 2,500,000, or from 500,000 to 2,500,000.

In some embodiments, the average read depth may be at least 50×, or 100× for the portion of the reference genome covered.

The sample can contain cancer cells and non-cancer cells. The presence of cancer cells and non-cancer cells in the sample can allow the methods of this invention to detect somatic mutations, as well as to distinguish somatic mutations from germline mutations without using a comparator sample such as a germline comparator sample.

In general, cancer cells may be present because the sample can be taken from a subject having cancer, and the sample may contain tissue or cells taken from a cancer situs. In some embodiments, the sample can be tissue or cells removed from a tumor. In certain embodiments, the sample can be tissue or cells removed from a malignancy. In further embodiments, the sample can be tissue or cells removed from a tumor, which includes a margin of non-tumor tissue or cells.

Embodiments of this invention include a unique algorithm used in methods for directly detecting somatic mutations and evaluating a tumor mutation burden using only a single sample from a subject, without a step for subtraction of germline quantities obtained from a comparator sample.

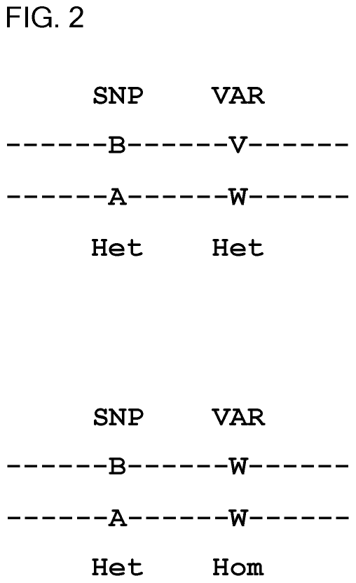

FIG. 2 shows an illustration of germline alleles and germline variants. In FIG. 2, top, is shown nucleic acid sequences in germline cells for a heterozygous variant position having alleles V and W, which is located near a heterozygous SNP having alleles B and A. Each SNP allele is associated with only one variant allele, i.e. BV and AW. In detecting these allele pairs, only two unique sequences detections are expected, BV and AW. In sequencing by fragmentation, for read lengths that cover both SNP and VAR positions, only two unique sequence reads are expected, BV and AW.

It can be noted in FIG. 2, top, that the probability of having both variant alleles V and W associated with B is extremely small to zero.

In FIG. 2, bottom, is shown nucleic acid sequences in germline cells for a homozygous variant position having alleles W and W, which is located near a heterozygous SNP having alleles B and A. Each SNP allele is associated with the same variant allele, i.e. BW and AW. In detecting these allele pairs, only two unique sequences detections are expected, BW and AW. In sequencing by fragmentation, for read lengths that cover both SNP and VAR positions, only two unique sequence reads are expected, BW and AW.

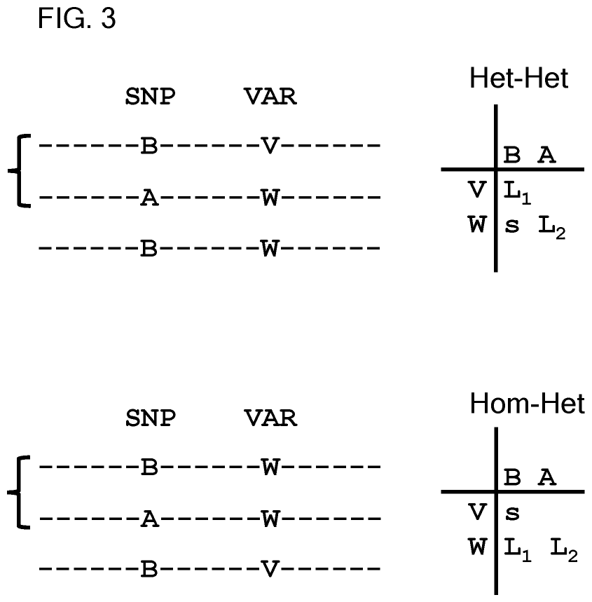

FIG. 3 shows an illustration of somatic alleles and somatic variants.

In FIG. 3, top, is shown nucleic acid sequences in sample cells for a heterozygous variant position having alleles V and W, which is located near a heterozygous SNP having alleles B and A. In cells without somatic mutation variant, each SNP allele would be associated with only one variant allele, e.g. BV and AW. In detecting these allele pairs, only two unique sequences detections are expected, BV and AW. In sequencing by fragmentation, for read lengths that cover both SNP and VAR positions, only two unique sequence reads are expected, BV and AW. Thus, there would be relatively large read counts L₁ and L₂ for the two normally expected allele pairs BV and AW. In cancer cells with a somatic mutation variant, a SNP allele would be associated with a second variant allele, e.g. BW. Thus, there would be relatively small read count s for the new allele pair BW. The presence of non-zero counts for s indicates that a SNP allele B is found or associated with two different variant alleles, V and W. Thus, either V or W can be taken as a de novo mutation, and more particularly a somatic mutation. The non-zero count for s indicates that BW arises from cancer cells by somatic mutation.

In FIG. 3, top, is shown a Het-Het count matrix for a heterozygous variant position having alleles V and W, which is located near a heterozygous SNP having alleles B and A. In the absence of cancer cells, or in the absence or somatic mutations, s is zero and FIG. 3, top, becomes equivalent to FIG. 2, top.

Embodiments of this invention contemplate a feature which is the Allele Ratio for somatic mutations. The Allele Ratio can be defined as a ratio of the non-wild type base, and can vary from 0 to 100%.

In general, the Allele Ratio describes the fraction of variant alleles relative to WT reference alleles, and can vary from 0 to 100%.

In general, an Allele Ratio of zero can be found if no cancer cells containing a somatic mutation are present. In general, an Allele Ratio of 100% would indicate that somatic mutations are present at a high level.

In FIG. 3, bottom, is shown nucleic acid sequences in sample cells for a homozygous variant position having alleles W and W, which is located near a heterozygous SNP having alleles B and A. In cells without somatic mutation variant, each SNP allele would be associated with only one variant allele, e.g. BW and AW. In detecting these allele pairs, only two unique sequences detections are expected, BW and AW. In sequencing by fragmentation, for read lengths that cover both SNP and VAR positions, only two unique sequence reads are expected, BW and AW. Thus, there would be relatively large read counts L₁ and L₂ for the two normally expected allele pairs BW and AW. In cancer cells with a somatic mutation variant, a SNP allele would be associated with a second variant allele, e.g. BV. Thus, there would be relatively small read count s for the new allele pair BV. The presence of non-zero counts for s indicates that a SNP allele B is found or associated with two different variant alleles, V and W. Thus, either V or W can be taken as a de novo mutation, and more particularly a somatic mutation. The non-zero count for s indicates that BV arises from cancer cells by somatic mutation.

In FIG. 3, bottom, is shown a Hom-Het count matrix for a homozygous variant position having alleles W and W, which is located near a heterozygous SNP having alleles B and A. In the absence of cancer cells, or in the absence or somatic mutations, s is zero and FIG. 3, bottom, becomes equivalent to FIG. 2, bottom.

The presence of non-zero s indicates that a SNP allele B is found or associated with two different variant alleles, V and W, and therefore identifies that a de novo mutation is present.

In some embodiments, for variants located near a heterozygous SNP, a third non-zero read count, detectable above noise level, can only arise from somatic mutations in cancer cells. The third significant read count can be obtained in the presence of non-cancer cells, and without subtraction of any germline quantities obtained from a second germline comparator sample. In fact, a second germline comparator sample is not needed in this unique algorithm.

### Tumor Mutation Burden

Without wishing to be bound by any particular theory, a method for evaluation of somatic mutation scores and tumor mutation burden (TMB) is set forth below.

TMB values according to this invention can be calculated using sequencing data obtained from a single sample from a subject using the unique algorithm of this invention that does not require germline subtraction. The sequencing data can be obtained by various methods known in the art including microelectrophoretic methods, sequencing by hybridization, real-time observation of single molecules, and cyclic-array sequencing.

TMB values can be calculated using fragmentation sequencing data obtained from a single sample from a subject using the unique algorithm of this invention that does not require germline subtraction. Only sequence reads having a length spanning both variant and SNP positions may be included in the assembly of a count matrix. In general, the read should cover the SNP and the position to be counted. Germline subtraction using a comparator sample is not necessary. A set of SNP positions can be used to obtain the sequencing data. The allele frequency of the SNP can be compared with the variant to determine whether the variant was germline or somatic.

A SNP region of about one read length can be used to detect a variant near a SNP position. The read length can be sufficient to cover both the SNP position and the variant position. A set of SNP regions can provide the sequencing data needed to detect somatic variants and quantify a value of TMB for a sample.

As used herein, a variant may be “near” a SNP position when the variant is within about one sequencing read length of the SNP position. A SNP region may be ±1 read length about a SNP position.

Examples of human SNP position sets known in the art include SNP Array 6.0 (Affymetrix).

For a SNP region including a variant position a count matrix can be calculated, where each element of the count matrix C(X1,X2) can be the number of mapped reads with non-SNP call X1=(T, C, G, or A) and SNP call X2=(T, C, G, or A).

The quantities X,Y and P,Q correspond to examples V,W and B,A respectively in FIGS. 2 and 3.

The two largest counts in this matrix, C(X,P)≥C(Y,Q), may be attributed to one of four position allele conditions:

HomHom: C(Y,Q)≤3 leaves only one significant count, C(X,P), which indicates that that both non-SNP and SNP positions were homozygous;

HetHom: X≠Y and P=Q, which indicates that the non-SNP position was heterozygous and the SNP position was homozygous;

HomHet: X=Y and P≠Q, which indicates that the non-SNP position was homozygous and the SNP position was heterozygous; and

HetHet: X≠Y and P≠Q, which indicates that both the non-SNP and SNP positions were heterozygous.

The HomHet and HetHet conditions with heterozygous SNP positions may be used to distinguish read counts attributable to somatic mutations from those attributable to normal germline allele pairings. For a sample from a subject having cancer, the somatic mutations can be attributed to presence of cancer cells. This can be done without separately obtaining germline comparator data from a separate sample.

For the count matrix described above, the presence of a third maximum count C(Z,P) or C(Z,Q) in the matrix can be attributed to a somatic mutation of a cancer cell.

The third maximum count can be used to detect a somatic mutation when the count is significantly above the background sequencing error rate. The average error rate, E, may be calculated from all other counts, except for the highest three counts. In certain embodiments, the average error rate, E, may be calculated from the average of all other counts in the matrix, except for the highest three counts.

A Phred-like significance score for a somatic mutation, which is a Chi-squared probability with one degree of freedom, may be calculated with Formula I:

S=(C(Z,P)²/(C(Z,P)+C(X,P))+(C(Z,P)−E)²/E)/2*10 Formula I

wherein C(Z,P) is the third element count, C(X,P) is the first element count, and E is an error rate calculated from the average of all other counts in the matrix, except for the highest three counts, for all SNP regions.

The value of the error rate E may be calculated as an average over all positions and is usually about 1 or less.

The TMB level can be taken as the number of positions having S>30, normalized by the total number of positions in the heterozygous SNP regions {N(HomHet)+N(HetHet)} in Mbases, as shown in Formula II:

TMB=N(S>30)/(N(HomHet)+N(HetHet))*1000000 Formula II

Without wishing to be bound by any particular theory, a method for determining a value for tumor mutation burden (TMB) based on the description above is set forth below.

TMB values can be calculated using fragmentation sequencing data obtained from a single sample from a subject using the unique algorithm of this invention that does not require germline subtraction. Germline subtraction using a comparator sample is not necessary. A set of SNP positions can be used.

The sequencing data from a set of SNP regions can be plotted to show the number of variant positions (y axis) versus the Allele Ratio (x axis). The area under the curve can be an estimate of the presence of somatic variants. Using this arrangement of the sequencing data, by integrating the area under the curve a value for the total number of variants that are identified as somatic variants can be obtained. The value for the total number of variants that are identified as somatic variants can be a measure of TMB. Thus, a measure of TMB can be obtained as the area under a curve from an Allele Ratio of about 15% up to an Allele Ratio of about 85%, or up to an Allele Ratio of about 65%, where the curve plots the number of variant positions (y axis) in a set of SNP regions against the Allele Ratio (x axis) of the variants.

In some embodiments, a measure of TMB can be obtained as the area under the variant count (y axis) Allele Ratio (x axis) curve from an Allele Ratio of about 15% up to an Allele Ratio of about 50%, or from an Allele Ratio of about 15% up to an Allele Ratio of about 55%, or from an Allele Ratio of about 15% up to an Allele Ratio of about 60%, or from an Allele Ratio of about 15% up to an Allele Ratio of about 65%, or from an Allele Ratio of about 15% up to an Allele Ratio of about 75%, or from an Allele Ratio of about 15% up to an Allele Ratio of about 85%.

In general, the somatic mutation occurrence in a position with non-wild type base may be rare, so the errors for the high allele ratio values may be less reliable. Thus, the area under the variant count (y axis) Allele Ratio (x axis) curve can preferably be taken from an Allele Ratio of about 15% up to an Allele Ratio of about 65% to reduce error.

In some embodiments, a measure of an average error rate, E, can be obtained as the value of the variant count (y axis) Allele Ratio (x axis) curve at an Allele Ratio of about 10-15%.

### Systems

In a system of this invention, results of sample analysis may be communicated to physicians, caregivers, genetic counselors, patients, and others in a transmittable form that can be communicated or transmitted to any of the above parties. Such a form can vary and can be tangible or intangible. The results can be embodied in descriptive statements, diagrams, photographs, charts, images or any other displayable forms. The statements and visual forms can be recorded on a tangible medium such as papers, computer readable media such as floppy disks, compact disks, etc., or on an intangible medium, e.g., an electronic medium in the form of email or website on internet or intranet. In addition, results can also be recorded in a sound form and transmitted through any suitable medium, e.g., analog or digital cable lines, fiber optic cables, etc., via telephone, facsimile, wireless mobile phone, internet phone and the like.

In a system of this invention, information and data of a test result can be produced anywhere, and transmitted to a different location. This invention further encompasses methods for producing a transmittable form of test information for at least one patient sample.

A computer-based analysis function can be implemented in any suitable language and/or browsers. For example, it may be implemented with C language and preferably using object-oriented high-level programming languages such as Visual Basic, SmallTalk, C++, and the like. The application can be written to suit environments such as the Microsoft Windows™ environment including Windows™ 98, Windows™ 2000, Windows™ NT, and the like. In addition, the application can also be written for the Maclntosh™, SUN™, UNIX or LINUX environment. In addition, the functional steps can also be implemented using a universal or platform-independent programming language. Examples of such multi-platform programming languages include, but are not limited to, hypertext markup language (HTML), JAVA™, JavaScript™, Flash programming language, common gateway interface/structured query language (CGI/SQL), practical extraction report language (PERL), AppleScript™ and other system script languages, programming language/structured query language (PL/SQL), and the like. Java™- or JavaScript™-enabled browsers such as HotJava™, Microsoft™ Explorer™, or Netscape™ can be used. When active content web pages are used, they may include Java™ applets or ActiveX™ controls or other active content technologies.

An analysis function can also be embodied in computer program products and used in the systems described above or other computer- or internet-based systems. Accordingly, another aspect of the present invention relates to a computer program product comprising a computer-usable medium having computer-readable program codes or instructions embodied thereon for enabling a processor to carry out somatic mutation score and/or TMB analysis. These computer program instructions may be loaded onto a computer or other programmable apparatus to produce a machine, such that the instructions which execute on the computer or other programmable apparatus create means for implementing the functions or steps described above. These computer program instructions may also be stored in a computer-readable memory or medium that can direct a computer or other programmable apparatus to function in a particular manner, such that the instructions stored in the computer-readable memory or medium produce an article of manufacture including instruction means which implement the analysis. The computer program instructions may also be loaded onto a computer or other programmable apparatus to cause a series of operational steps to be performed on the computer or other programmable apparatus to produce a computer implemented process such that the instructions which execute on the computer or other programmable apparatus provide steps for implementing the functions or steps described above.

Embodiments of this invention can provide a non-transitory machine-readable storage medium having stored therein instructions for execution by a processor which cause the processor to perform the steps of a method for determining and calculating TMB.

Examples of a non-volatile, non-transitory machine-readable storage medium include various kinds of read only memory (ROM), hard drives, solid state memory devices, flash drives, compact disc read only memory (CD-ROM), DVDs, optical disks, magnetic disks, or any other storage media which may be used to carry or store program code having computer-executable instructions or data structures. The media may be accessed by a general purpose or special purpose computer, such as a processor.

Embodiments of this invention may provide a computing system, which may have one or more processors, one or more memory devices, a file system, a communication module, an operating system, and/or a user interface, each of which can be communicatively coupled.

A computing system can have an operating system, which may be arranged to utilize various hardware and software resources. An operating system can be arranged to receive and execute instructions for other components of the system.

Examples of computing systems include laptop computers, desktop computers, server computers, mobile phones or smartphones, tablets, and other portable computing systems.

Examples of a computing system include a processor, a special-purpose, or a general-purpose computer.

A processor may be arranged to execute instructions stored on a machine-readable storage medium. A processor may include a one or more microprocessors, various controllers, a digital signal processor, or an application-specific integrated circuit, and can receive and/or transfer data, as well as execute stored instructions to transform the data. In some embodiments, a processor may receive, interpret, and execute instructions from program code or various media. A processor can receive and transform data, as well as store data in a memory, or file. In certain embodiments, a processor can fetch instructions from a memory or file and receive an instruction into a memory.

A machine-readable storage medium can be non-volatile. A memory or medium can store instruction or data files in a file system and can include a machine-readable storage medium. A machine-readable storage medium can be non-transitory. A machine-readable storage medium can have stored therein instructions which can be executable by a processor.

A communication device can be any apparatus, system, or combination of components which can transmit and/or receive data. Data can be transmitted and/or received via a network, or a communication line. A communication device may be communicatively linked to other components.

Examples of communication devices include a network card, a modem, an antenna, an infrared or visible communication component, a Bluetooth component, a communication chipset, a wide area network, a WiFi component, an 802.6 or higher device, and a cellular communication device. A communication device can exchange data over a line, wire or network to other components, devices or systems.

A system of this disclosure can include one or more processors, one or more non-transitory machine-readable storage media, one or more file systems, one or more memory devices, an operating system, one or more communication modules, and one or more user interfaces, each of which may be communicatively linked.

Some computational biology methods are described in, for example, Setubal et al., Introduction To Computational Biology Methods (1997); Salzberg et al., Computational Methods In Molecular Biology (1998); Rashidi & Buehler, Bioinformatics Basics: Application In Biological Science And Medicine (2000); Ouelette & Bzevanis, Bioinformatics: A Practical Guide For Analysis Of Gene And Proteins (2001).

### Anticancer Agents

Immune checkpoint inhibitor drugs can unleash T cells to kill cancer cells in a subject. These drugs can block proteins which enable cancer cells to evade the immune system and improve survival rates.

Immune checkpoint inhibitors are therapeutic agents which can prevent or inhibit immune cells and/or the immune response from being turned off, or down-regulated or inhibited by the very cancer cells intended to be killed.

In general, immune checkpoint inhibitor drugs are effective for less than 13% of subjects having cancer. Thus, it is useful to be able to select and identify subjects who benefit from treatment with such drugs.

Examples of immune checkpoint inhibitors include PD1 inhibitors, ipilimumab (see, e.g., Gulley & Dahut, Nat. Clin. Practice Oncol. (2007) 4:136-137), tremelimumab (see, e.g., Ribas et al., Oncologist (2007) 12:873-883), and the agents listed in Table 1.

TABLE 1
Checkpoint inhibitor agents

| Drug | Target | Uses |
|---|---|---|
| Yervoy (ipilimumab, | CTLA4 | Melanoma, NSCLC, |
| MDX-010, MDX-101) | | SCLC, bladder cancer, |
| (Bristol-Myers Squibb) | | prostate cancer |
| Tremelimumab (ticilimumab, | CTLA4 | Mesothelioma |
| CP-675, 206) (AstraZeneca) | | |
| Opdivo (nivolumab) | PD1 | Malignant melanoma |
| (Bristol-Myers Squibb) | | |
| Keytruda (pembrolizumab, | PD1 | Malignant melanoma |
| lambrolizumab, MK-3475) | | |
| (Merck) | | |
| MEDI4736 | PDL1 | NSCLC |
| (AstraZeneca) | | |
| MPDL3280A | PDL1 | Urothelial bladder |
| (Roche/Genentech) | | cancer or NSCLC |
| Pidilizumab (CT-011) | PD1 | Hematologic or |
| (CureTech) | | solid tumors |
| lirilumab (BMS-986015) | KIR | Hematologic or |
| (Bristol-Myers Squibb) | | solid tumors |
| Indoximod (NLG-9189) | IDO1 | Breast cancer |
| (Newlink Genetics) | | |
| INCB024360 | IDO1 | Solid tumors |
| (Incyte) | | |
| MEDI0680 (AMP-514) | PD1 | Solid tumors |
| (AstraZeneca) | | |
| MSB-0010718C | PDL1 | Solid tumors |
| (Merck KGaA) | | |
| PF-05082566 | 4-1BB | Hematologic or |
| (Pfizer) | (CD137) | solid tumors |
| MEDI6469 | OX40 | Solid tumors |
| (AstraZeneca) | (CD134) | |
| BMS-986016 | LAG3 | Hematologic or |
| (Bristol-Myers Squibb) | | solid tumors |
| NLG-919 | IDO1 | Solid tumors |
| (Newlink Genetics) | | |
| Urelumab (BMS-663513) | 4-1BB | Hematologic or |
| (Bristol-Myers Squibb) | (CD137) | solid tumors |

### Additional Definitions

The following terms or definitions are provided solely to aid in the understanding of the disclosure.

Unless specifically defined herein, all terms used herein have the same meaning as they would to one skilled in the art of the present disclosure.

Some methods are given in Sambrook et al., Molecular Cloning: A Laboratory Manual, 2^{nd} ed., Cold Spring Harbor Press, Plainview, N.Y. (1989); and Ausubel et al., Current Protocols in Molecular Biology (Supplement 47), John Wiley & Sons, New York (1999).

Unless expressly defined otherwise herein, the terms used herein should not be construed to have a scope less than understood by a person of ordinary skill in the art.

As used herein, a “single nucleotide polymorphism” (SNP) or “SNP locus” is a locus with alleles that differ at a single base, with the rarer allele having a frequency of at least 1% in a population.

As used herein, the “alleles” at a genetic locus are the set of all genetic variants that occur at that locus in a population, each variant being a single “allele.” For example, there are generally only two alleles at a SNP locus.

As used herein, a “variant” is a difference between a test genetic sequence and a reference genetic sequence. A variant may differ at a single base, or a variant may differ at more than one base. Variants also include insertions and deletions.

As used herein, a first variant is “linked” to a second variant if the first and second variant are both located on the same chromosomal (maternal or paternal) DNA strands. “Linkage” refers to the state of two or more variants being linked.

A “position allele model” is a model that represents the linkage between the alleles at a test locus and the alleles at a SNP locus. In the germline, the position allele model will typically describe linkage between the paternal allele at the test locus and the paternal allele at the SNP locus, as well as linkage between the maternal allele at the test locus and the maternal allele at the SNP locus. In cases where a somatic variant is present at the test locus (i.e. a third possible allele at the test locus), the position allele model will additionally describe linkage between this third allele at the test locus and either the maternal or paternal allele at the SNP locus.

As used herein, “mutation” is described in detail below, but generally refers to an acquired nucleotide change in a somatic tissue as compared to a subject's germline. “Mutation load” is described in detail below, but generally refers to the number or proportion of analyzed loci harboring a mutation, with “high mutation load” or “HML” generally referring to a number or proportion, or score derived therefrom, that exceeds some reference or threshold.

As used herein, “next generation sequencing” or “NGS” refers to a variety of high-throughput sequencing processes and technologies that parallelize the sequencing process, producing thousands or millions of sequences at once. NGS is generally conducted with the following steps: First, DNA sequencing libraries are generated by clonal amplification by PCR in vitro; second, the DNA is sequenced by synthesis, such that the DNA sequence is determined by the addition of nucleotides to the complementary strand rather through chain-termination chemistry typical of Sanger sequencing; third, the spatially segregated, amplified DNA templates are sequenced simultaneously in a massively parallel process, typically without the requirement for a physical separation step. NGS parallelization of sequencing reactions can generate hundreds of megabases to gigabases of nucleotide sequence reads in a single instrument run. Unlike conventional sequencing techniques, such as Sanger sequencing, which typically report the average genotype of an aggregate collection of molecules, NGS technologies typically digitally tabulate the sequence of numerous individual DNA fragments (sequence reads discussed in detail below), such that low frequency variants (e.g., variants present at less than about 10%, 5% or 1% frequency in a heterogeneous population of nucleic acid molecules) can be detected. The term “massively parallel” can also be used to refer to the simultaneous generation of sequence information from many different template molecules by NGS.

NGS strategies can include several methodologies, including, but not limited to: (i) microelectrophoretic methods; (ii) sequencing by hybridization; (iii) real-time observation of single molecules, and (iv) cyclic-array sequencing. Cyclic-array sequencing refers to technologies in which a sequence of a dense array of DNA is obtained by iterative cycles of template extension and imaging-based data collection. Commercially available cyclic-array sequencing technologies include, but are not limited to 454 sequencing, for example, used in 454 Genome Sequencers (Roche Applied Science; Basel), Solexa technology, for example, used in the Illumina Genome Analyzer, Illumina HiSeq, MiSeq, and NextSeq (San Diego, Calif.), the SOLiD platform (Applied Biosystems; Foster City, Calif.), the Polonator (Dover/Harvard) and HeliScope Single Molecule Sequencer technology (Helicos; Cambridge, Mass.). Other NGS methods include single molecule real time sequencing (e.g., Pacific Bio) and ion semiconductor sequencing (e.g., Ion Torrent sequencing). See, e.g., Shendure & Ji, Next Generation DNA Sequencing, NAT. BIOTECH. (2008) 26:1135-1145 for a more detailed discussion of NGS sequencing technologies.

As used herein, “patient” or “individual” or “subject” refers to a human. A patient, individual or subject can be male or female. A patient, individual or subject can be one who has already undergone, or is undergoing, a therapeutic intervention for disease. A patient, individual or subject can also be one who has not been previously diagnosed with a disease.

As used herein, “sample” or “biological sample” refers to samples such as biopsy or tissue samples, frozen samples, blood and blood fractions or products (e.g., serum, platelets, red blood cells, and the like), tumor samples, sputum, bronchoalveolar lavage, cultured cells, e.g., primary cultures, explants, and transformed cells, stool, urine, etc.

A “biopsy” refers to the process of removing a tissue sample for diagnostic or prognostic evaluation, and to the tissue specimen itself. Various biopsy techniques can be applied to the methods of the present disclosure. The biopsy technique applied will depend on the tissue type to be evaluated (e.g., lung, etc.), the size and type of the tumor, among other factors. Representative biopsy techniques include, but are not limited to, excisional biopsy, incisional biopsy, needle biopsy, surgical biopsy, and bone marrow biopsy. An “excisional biopsy” refers to the removal of an entire tumor mass with a small margin of normal tissue surrounding it. An “incisional biopsy” refers to the removal of a wedge of tissue that includes a cross-sectional diameter of the tumor. A diagnosis made by endoscopy or fluoroscopy can require a “core-needle biopsy”, or a “fine-needle aspiration biopsy” which generally obtains a suspension of cells from within a target tissue.

A “bodily fluid” include all fluids obtained from a mammalian body, either processed (e.g., serum) or unprocessed, which can include, for example, blood, plasma, urine, lymph, gastric juices, bile, serum, saliva, sweat, and spinal and brain fluids. A biological sample is typically obtained from a subject.

As used herein, “cancer cell samples” or “tumor sample” means a specimen comprising either at least one cancer cell or biomolecules derived therefrom. Examples of cancer include lung cancer (e.g., non-small cell lung cancer (NSCLC)), ovarian cancer. colorectal cancer, breast cancer, endometrial cancer, and prostate cancer. Non-limiting examples of such biomolecules include nucleic acids and proteins. Biomolecules “derived” from a cancer cell sample include molecules located within or extracted from the sample as well as artificially synthesized copies or versions of such biomolecules. One illustrative, non-limiting example of such artificially synthesized molecules includes PCR amplification products in which nucleic acids from the sample serve as PCR templates. “Nucleic acids of” a cancer cell sample include nucleic acids located in a cancer cell or biomolecules derived from a cancer cell.

As used herein, “score” means a value or set of values selected so as to provide a quantitative measure of a variable or characteristic of a subject's condition or the degree of mutation load in a sample, and/or to discriminate, differentiate or otherwise characterize mutation load. The value(s) comprising the score can be based on, for example, quantitative data resulting in a measured amount of one or more sample constituents obtained from the subject. In certain embodiments the score can be derived from a single constituent, parameter or assessment, while in other embodiments the score is derived from multiple constituents, parameters and/or assessments. The score can be based upon or derived from an interpretation function; e.g., an interpretation function derived from a particular predictive model using any of various statistical algorithms. A “change in score” can refer to the absolute change in score, e.g. from one time point to the next, or the percent change in score, or the change in the score per unit time (i.e., the rate of score change).

As used herein, a “test locus” is a genomic locus (e.g., single nucleotide at a specified position within a chromosome) whose sequence or genotype is assessed according to the present disclosure, wherein a mutation at such a locus (e.g., as compared to a reference genotype or sequence) is potentially counted in a measurement of mutation load.

As used herein, the term “treatment” or “therapy” or “therapeutic regimen” includes all clinical management of a subject and interventions, whether biological, chemical, physical, or a combination thereof, intended to sustain, ameliorate, improve, or otherwise alter the condition of a subject. These terms may be used synonymously herein. Treatments include but are not limited to administration of prophylactics or therapeutic compounds (including small molecule and biologic drugs), exercise regimens, physical therapy, dietary modification and/or supplementation, bariatric surgical intervention, administration of therapeutic compounds (prescription or over-the-counter), and any other treatments efficacious in preventing, delaying the onset of, or ameliorating disease characterized by HML. A “response to treatment” includes a subject's response to any of the above-described treatments, whether biological, chemical, physical, or a combination of the foregoing. A “treatment course” relates to the dosage, duration, extent, etc. of a particular treatment or therapeutic regimen. An initial therapeutic regimen as used herein is the first line of treatment.

Additional Aspects of the Disclosure

Aspects of this Disclosure Include the Following:

Methods for detecting the presence of a somatic variant at a test locus in a sample, comprising: detecting on a first contiguous strand of nucleic acid from the sample a first allele at a single nucleotide polymorphism (“SNP”) locus, and a second allele at the test locus; detecting on a second contiguous strand of nucleic acid from the sample a third allele at the SNP locus and a fourth allele at the test locus; and detecting on a third contiguous strand of nucleic acid from the sample, the third allele at the SNP locus and a fifth allele at the test locus, wherein the first allele and the third allele are different alleles, and the fourth allele and the fifth allele are different alleles.

In some embodiments, the second allele and the fourth allele are the same or different alleles. The nucleic acid can be deoxyribonucleic acid (DNA). One or more alleles may be detected by sequencing. One or more alleles may be detected by hybridization. One or more alleles may be detected by polymerase chain reaction (PCR) amplification. The sample may comprise a cell with a somatic variant at the test locus, and a cell without a somatic variant at the test locus. The sample may be a tissue sample. The sample may be a tumor sample.

Methods for detecting a somatic variant in a sample, comprising: detecting a SNP locus at which the individual is heterozygous; detecting at a test position within a contiguous region surrounding the SNP locus a first test allele linked to a first SNP allele at the SNP locus; and detecting at the test position within the contiguous region surrounding the SNP locus a second test allele linked to the first SNP allele at the SNP locus, wherein the first test allele and the second test allele are different alleles. In some embodiments, further comprising identifying at the test position within the contiguous region surrounding the SNP locus a third test allele linked to a second SNP allele at the SNP locus, wherein the first SNP allele and the second SNP allele are different alleles. The first test allele and third test allele may be the same allele. The first test allele and third test allele may be different alleles. The one or more alleles may be detected by sequencing, hybridization, or by polymerase chain reaction amplification. The sample may comprise a cell with a somatic variant at the test locus, and a cell without a somatic variant at the test locus. The sample may be a tissue sample. The sample may be a tumor sample.

Methods for measuring the frequency of somatic variants in a sample, comprising: detecting a plurality of SNP loci at which the sample is heterozygous; within a contiguous region surrounding each SNP locus identified in part a, assaying a plurality of test loci to detect a number of test alleles linked to each SNP allele for each of the plurality of test loci; and determining a variant frequency, comprising the number of test loci where the detected number of test alleles linked to a SNP allele is greater than one, normalized to the total number of test loci assayed. The one or more alleles may be detected by sequencing, by hybridization, or by polymerase chain reaction amplification. The sample may comprise a cell with a somatic variant at the test locus, and a cell without a somatic variant at the test locus. The sample may be a tissue sample, or a tumor sample.

Systems for detecting somatic mutations, comprising a plurality of sensors for measuring a position allele model number for each position in a region surrounding each of a predetermined set of SNPs.

Methods for treating an individual with an immune checkpoint inhibitor, comprising: detecting a plurality of SNP loci at which the individual is heterozygous; within a contiguous region surrounding each SNP locus identified in part a, assaying a plurality of test loci to detect a number of test alleles linked to each SNP allele for each of the plurality of test loci; determining a variant frequency, comprising the number of test loci where the detected number of test alleles linked to a SNP allele is greater than one, normalized to the total number of test loci assayed; and administering to the individual a therapeutically effective amount of an immune checkpoint inhibitor when the variant frequency exceeds a predetermined threshold. The one or more alleles may be detected by sequencing, by hybridization, or by polymerase chain reaction amplification. The sample may comprise a cell with a somatic variant at the test locus, and a cell without a somatic variant at the test locus. The sample may be a tissue sample, or a tumor sample.

All publications, patents and literature specifically mentioned herein are hereby incorporated by reference in their entirety for all purposes.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In addition, the materials, methods, and examples herein are illustrative only and not intended to be limiting.

Although the foregoing disclosure has been described in some detail by way of illustration and examples for purposes of clarity of understanding, it will be understood by persons of skill in the art that various changes and modifications may be practiced within the scope of the invention and the appended claims.

### EXAMPLES

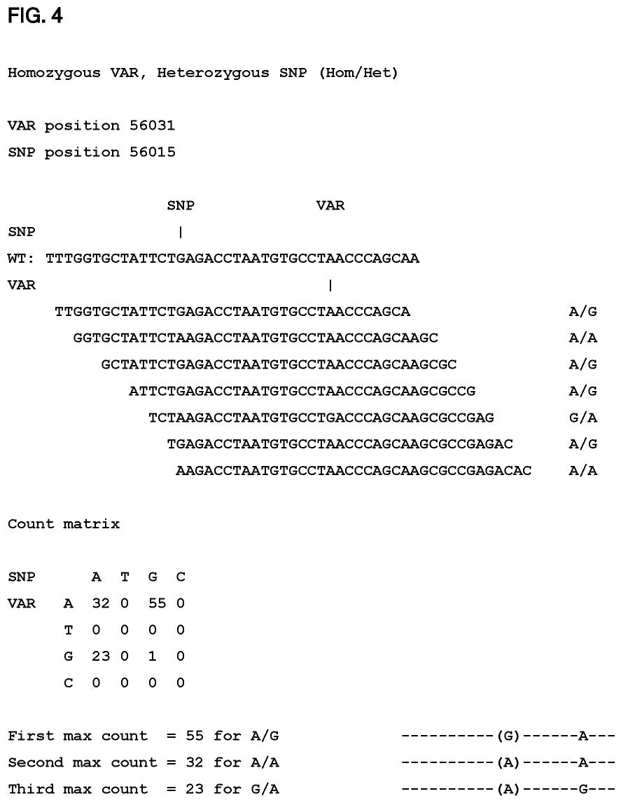

Example 1: FIG. 4 shows results of a method for detecting and evaluating tumor mutation burden by nucleic acid sequencing. For a model comprising a homozygous somatic variant located near a heterozygous SNP (Hom/Het), a sequence read stack was mapped to a reference genome (WT) as shown. A count matrix was assembled which showed the detection of allele pairs GA (55), AA (32), and AG (23). The appearance of the third maximum count AG (23) arose from somatic mutations in cancer cells.

The Allele Ratio was calculated as a ratio of different alleles in the VAR position. In this Hom-Het example, the Allele Ratio=(23+1)/(32+55+23+1)*100=21.6%.

The SNP was heterozygous with an Allele Ratio (32+23)/{(32+23)+(55+1)}×100=49.5% (A/G 55:56).

The error rate E, as shown in FIG. 4, was about 1.0. Thus, the value for S was about S=((23×23/(23+55))+(23−E)(23−E)/E)/2×10=2679. The value of E was calculated as an average over all positions, and was typically about 1.0 or less.

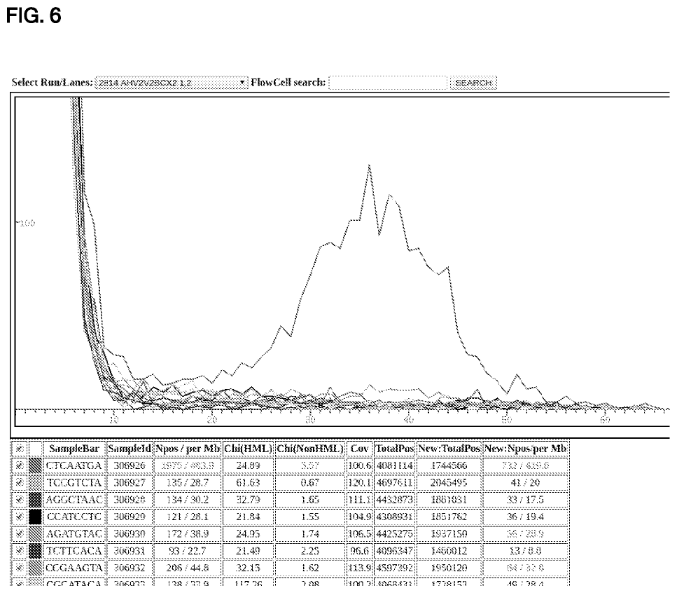

For this example position, the sample was 306926 in FIG. 6, having high TMB.

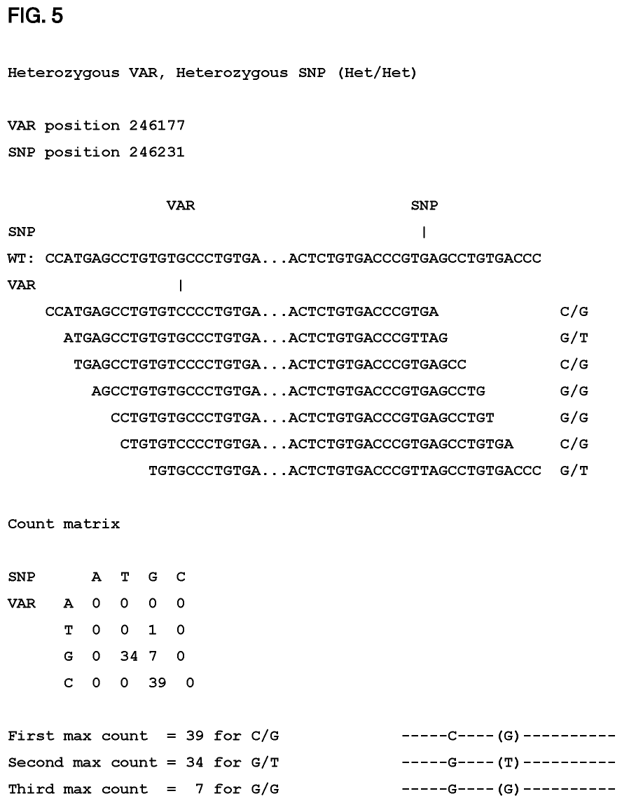

Example 2: FIG. 5 shows results of a method for detecting and evaluating tumor mutation burden by nucleic acid sequencing.

In this particular example, the read length was 100 bp, and the total SNP window was 100*2−1=199 bp. For this example position, the sample was 306926 in FIG. 6, having high TMB.

For a model comprising a heterozygous somatic variant located near a heterozygous SNP (Het/Het), a count matrix was assembled which showed the detection of alleles CG (39), GT (34), and GG (7). The appearance of the third maximum count GG (7) arose from somatic mutations in cancer cells.

The Allele Ratio was calculated as a ratio of different alleles in the VAR position. In this Het-Het example, Allele Ratio=39/(34+7+39)*100=48.8%.

The SNP was heterozygous as T/G.

Example 3: FIG. 6 shows sequencing data from colon cancer samples. Each curve represents the number of variant positions (Y axis) by allele ratio % (X axis). One sample showed a large peak representing a high-TMB sample. The tall peak on the left side at very low allele ratio values, less than 10%, reflects sequencing errors which are ignored. For counting the TMB score, the TMB count was taken as the area under the curve in the range of Allele Ratios from 15% to 65%. Data from FIG. 6 are shown in Table 2. The last two columns of Table 2 show the total number of qualified positions and the TMB values, absolute and normalized per 1 Mb. Sample 306926 has TMB of 417 per Mb, and sample 306932 has TMB of 32.7 per Mb.

TABLE 2
TMB (PerMb) for colon cancer samples

| SampleTag | SampleID | Coverage | TotalPos | MutPos | PerMb |
|---|---|---|---|---|---|
| CTCAATGA | 306926 | 100.3 | 1720440 | 717 | 416.8 |
| TCCGTCTA | 306927 | 119.9 | 2019276 | 40 | 19.8 |
| AGGCTAAC | 306928 | 110.8 | 1856679 | 32 | 17.2 |
| CCATCCTC | 306929 | 104.7 | 1830688 | 36 | 19.7 |
| AGATGTAC | 306930 | 106.1 | 1913312 | 56 | 29.3 |
| TCTTCACA | 306931 | 96.4 | 1459685 | 13 | 8.9 |
| CCGAAGTA | 306932 | 113.7 | 1926863 | 63 | 32.7 |
| CGCATACA | 306933 | 100.0 | 1706073 | 49 | 28.7 |
| AATGTTGC | 306934 | 128.8 | 2076785 | 23 | 11.1 |
| TGAAGAGA | 306935 | 115.8 | 1904586 | 52 | 27.3 |
| AGATCGCA | 306936 | 97.3 | 1774434 | 29 | 16.3 |
| AAGAGATC | 306937 | 124.3 | 2087068 | 44 | 21.1 |
| CAACCACA | 306938 | 139.7 | 2174624 | 44 | 20.2 |
| TGGAACAA | 306939 | 155.4 | 2123021 | 30 | 14.1 |
| CCTCTATC | 306940 | 133.8 | 2152846 | 16 | 7.4 |
| ACAGATTC | 306941 | 118.9 | 2049170 | 55 | 26.8 |

TotalPos = number of selected positions with coverage 50 or more
MutPos = number of variant positions with score 30 or more
PerMb = MutPos * 1000000 / TotalPos

In general, TMB having 10 mutations per Mb is relatively high and corresponds to a total of over 32,000 somatic mutations when extrapolated to the whole genome.

Referring to FIG. 6, the TMB was calculated from positions with the mutation score 30 or more and with the allele ratio in the range 15-65% were counted and normalized by the total number of qualified positions in Mb. Referring to FIG. 6, the data curve showed the number of variant positions (Y axis) having the required score.

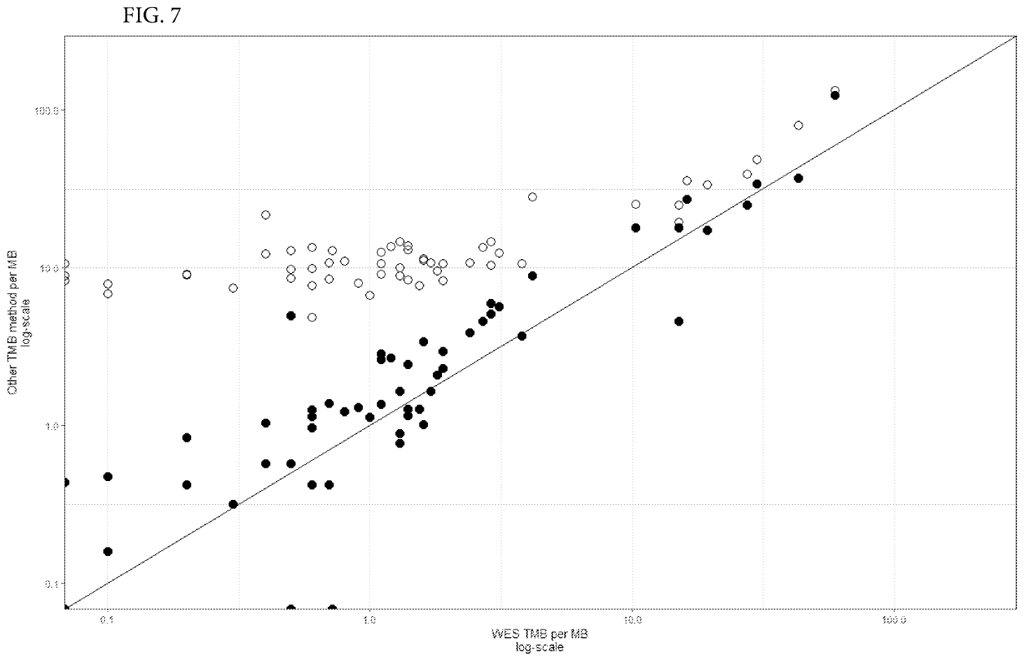

Example 4: FIG. 7 shows a plot of data obtained using a SNP-based method of this invention for detecting and evaluating tumor mutation burden in colon and breast cancer samples by nucleic acid sequencing as compared to conventional methods involving subtracting data from a germline comparator sample or germline filtering. The data from FIG. 7 is recapitulated in Table 3.

The samples for colon cancer were Colon Micro-Satellite. The samples for breast cancer were a set of 44 patient samples, which were platinum sensitive breast tumor.

TABLE 3
Comparison of TMB analysis of this
invention to conventinal methods

| | | | | Y axis | Y axis |
|---|---|---|---|---|---|
| | | | | (open | (filled |
| No. | Sampled | Cohort | X axis | circles) | circles) |
| 1 | 172326 | breast | 0 | 8.85 | 0.433243 |
| 2 | 172327 | breast | 0.5 | 12.85 | 4.927275 |
| 3 | 172328 | breast | 1.1 | 9.05 | 1.353341 |
| 4 | 172332 | breast | 0.9 | 7.95 | 1.295587 |
| 5 | 172333 | breast | 0.4 | 12.2 | 1.032044 |
| 6 | 172336 | breast | 0.6 | 7.7 | 1.142761 |
| 7 | 172337 | breast | 1.1 | 10.55 | 2.612515 |
| 8 | 172339 | breast | 3.1 | 12.35 | 5.639995 |
| 9 | 172340 | breast | 0.1 | 7.85 | 0.475758 |
| 10 | 172341 | breast | 0.1 | 6.8 | 0.159636 |
| 11 | 172342 | breast | 1.7 | 10.7 | 1.649034 |
| 12 | 172345 | breast | 1.8 | 9.5 | 2.091111 |
| 13 | 172346 | breast | 1.6 | 11.35 | 1.014355 |
| 14 | 172347 | breast | 0.4 | 21.65 | 0.573091 |
| 15 | 172349 | breast | 0.2 | 9 | 0.834013 |
| 16 | 172350 | breast | 1.9 | 10.55 | 2.945048 |
| 17 | 172351 | breast | 0.3 | 7.4 | 0.31697 |
| 18 | 172352 | breast | 0.2 | 9.05 | 0.421089 |
| 19 | 172353 | breast | 0.7 | 8.4 | 0.419443 |
| 20 | 172354 | breast | 0.6 | 13.45 | 0.418599 |
| 21 | 172355 | breast | 0.5 | 9.75 | 0.569258 |
| 22 | 172356 | breast | 1 | 6.65 | 1.125821 |
| 23 | 172357 | breast | 1.6 | 11.1 | 3.386773 |
| 24 | 172358 | breast | 1.4 | 13.75 | 1.146581 |
| 25 | 172359 | breast | 1.4 | 8.35 | 1.268059 |
| 26 | 172360 | breast | 0.7 | 10.65 | 1.379488 |
| 27 | 172712 | breast | 3.8 | 10.55 | 3.698196 |
| 28 | 172713 | breast | 0.6 | 4.85 | 1.254093 |
| 29 | 172716 | breast | 15.1 | 19.425 | 4.567614 |
| 30 | 172719 | breast | 1.2 | 13.65 | 2.66069 |
| 31 | 172720 | breast | 0 | 8.2 | 0 |
| 32 | 172721 | breast | 1.3 | 14.65 | 0.890209 |
| 33 | 172722 | breast | 0.8 | 10.9 | 1.226617 |
| 34 | 172723 | breast | 2.7 | 13.35 | 4.582397 |
| 35 | 172724 | breast | 0 | 10.6 | 0 |
| 36 | 172727 | breast | 0.6 | 9.8 | 0.965028 |
| 37 | 172728 | breast | 2.4 | 10.7 | 3.881554 |
| 38 | 172729 | breast | 0.5 | 8.525 | 0 |
| 39 | 172730 | breast | 1.9 | 8.2 | 2.296721 |
| 40 | 173206 | breast | 1.4 | 12.925 | 2.432384 |
| 41 | 173207 | breast | 2.9 | 10.325 | 5.095719 |
| 42 | 173208 | breast | 1.3 | 9.975 | 1.652989 |
| 43 | 173210 | breast | 1.1 | 12.45 | 2.850926 |
| 44 | 175917 | breast | 1.3 | 8.9 | 0.767679 |
| 45 | 193406 | colon | 4.173179 | 27.86667 | 8.897859 |
| 46 | 193411 | colon | 59.46998 | 132.8667 | 123.6433 |
| 47 | 193412 | colon | 2.884223 | 14.55 | 5.940877 |
| 48 | 193413 | colon | 1.538395 | 7.7 | 1.260531 |
| 49 | 193415 | colon | 10.2934 | 25.1 | 17.85718 |
| 50 | 193416 | colon | 27.47211 | 38.96 | 24.94902 |
| 51 | 193417 | colon | 19.32901 | 33.43333 | 17.20717 |
| 52 | 193418 | colon | 15.11196 | 24.95 | 17.73474 |
| 53 | 193419 | colon | 29.84983 | 48.05 | 34.01409 |
| 54 | 193420 | colon | 16.15368 | 35.62 | 27.02036 |
| 55 | 271207 | colon | 0.719131 | 12.8 | 0 |
| 56 | 271208 | colon | 43.15642 | 79.3 | 36.93433 |

Using the direct SNP-based method of this invention (FIG. 7, filled circles) with only a tumor sample, and without a second germline comparator sample, an evaluation of tumor mutation burden was obtained that was surprisingly superior to conventional methods. The sensitivity of the SNP-based method of this invention (FIG. 7, filled circles) was surprisingly increased over the conventional methods.

In FIG. 7, open and filled circles at the same x-axis position represent measurements on the same patient sample by the method of this invention (FIG. 7, filled circles) as compared to germline filtering (FIG. 7, open circles).

In FIG. 7, the X-axis represents the TMB value that was assessed by whole exome sequencing where the germline variants were subtracted using a blood-based germline reference sample for each patient. The same samples were used for the whole exome sequencing as for the method of this invention (FIG. 7, filled circles) and the method of germline filtering (FIG. 7, open circles). This method is considered the conventional “gold standard” for which blood-based subtraction removes germline variants.

In FIG. 7, the Y-Axis shows how the method of this invention (FIG. 7, filled circles) and the method of germline filtering (FIG. 7, open circles) compared to the conventional “gold standard” approach. The Y-Axis values were determined from data obtained using an HRD assay.

More particularly, the SNP-based method of this invention (FIG. 7, filled circles) was surprisingly more accurate than a method of nucleic acid sequencing for evaluating tumor mutation burden using a database of known germline variants and filtering of common variants to attempt to remove germline background (FIG. 7, open circles). This conventional method for detecting and evaluating tumor mutation burden by nucleic acid sequencing using a database of known germline variants and filtering of common variants to attempt to remove germline background (FIG. 7, open circles) provided inaccurate tumor mutation burden levels. Thus, the accuracy and sensitivity of the unique and direct SNP-based method of this invention (FIG. 7, filled circles) was surprisingly increased and unexpectedly advantageous over methods attempting to subtract germline quantities (FIG. 7, open circles).

Further, the direct SNP-based method of this invention was surprisingly superior to conventional whole exome sequencing performed with germline subtraction over a wide range of mutation frequency from 0.1 mutations per Mb up to 100 mutations per Mb (1000-fold increase) because the direct SNP-based method of this invention did not require a germline subtraction sample and improved sensitivity. More particularly, the SNP-based method of this invention (FIG. 7, filled circles) did not utilize, and did not require paired tumor and germline comparator samples to subtract germline quantities. The SNP-based method of this invention (FIG. 7, filled circles) utilized only a tumor sample. The SNP-based method of this invention, using only a tumor sample, surprisingly detected, identified and separated somatic mutations from germline quantities.

More particularly, FIG. 7 shows that the SNP-based method of this invention (FIG. 7, filled circles) provided more concordant results to Whole Exome Sequencing (represented as the x-axis) than germline filtering (FIG. 7, open circles). As shown in FIG. 7, the method of germline filtering (FIG. 7, open circles) was inaccurate (diverged from the line) at about 10 TMB per megabase, or about 20 per megabase. Thus, germline filtering cannot accurately assess TMB values below about 10 per megabase, or even below about 20 per megabase.

Example 5: The method of this invention using a unique algorithm for directly detecting somatic mutations and evaluating a tumor mutation burden using only a first, single sample from a subject having cancer, without a step for subtraction of germline quantities, was compared to a method of whole exome sequencing (WES) using paired tumor and germline comparator samples to subtract germline quantities. The method of this invention was further compared to a MYCHOICE HRD-PLUS method with subtraction of a germline comparator.

Each of the WES and MYCHOICE HRD-PLUS methods were performed on matched tumor and normal DNA from 44 breast and 12 colon tumors. The MYCHOICE HRD-PLUS assay combines homologous recombination deficiency analysis with resequencing of 108 genes and MSI analysis.

For one comparison, a TMB measure was calculated from WES by identifying all variants in the paired samples, and subtracting the germline variants.

For a different comparison, the MYCHOICE HRD-PLUS was used. This assay targets about 27,000 SNPs distributed across the genome. Sequence reads of about 100 bp were mapped to the set of SNP segments with a ±400-base window around each SNP, and with a maximum of 7 mismatches.

Several error filters were applied to the mapped sequences to reduce potential ambiguity in mutation calls:

reads with multiple map locations were ignored;

read ends can be prone to sequencing errors, so bases 1-10 and >86 in each read were ignored;

if both forward (F) and reverse (R) reads of same insert were mapped, their map locations must correspond to the insert size of 50-500 bp;

either F or R reads must overlap SNP position;

if F and R reads overlap, their calls were combined, and in this case,

the SNP calls must be the same;

positions in the overlap with different base calls are ignored (identifiable sequencing error).

TMB values were calculated using the MYCHOICE HRD-PLUS data in two ways. First, with substraction of germline quantities. In this method, a 400 bp sequence adjacent to each SNP was observed. Variants were identified within these sequence regions, and then germline subtraction was performed using the paired samples.

In a second experiment, TMB values were calculated for the MYCHOICE HRD-PLUS data using only a first, single sample from a subject having cancer and the unique algorithm of this invention that does not require germline subtraction.

In the second experiment, only sequence reads spanning both the variant and SNP were included in the assembly of a count matrix. The allele frequency of the SNP was compared with the variant to determine whether the variant was germline or somatic. Germline subtraction was not used.

In this second experiment, for all remaining positions, a count matrix was calculated, where each element C(X1,X2) was the number of mapped reads with non-SNP call X1=(T, C, G, or A) and SNP call X2=(T, C, G, or A). The two largest counts in this matrix, C(X,P)≥C(Y,Q), were attributed to one of four position allele conditions:

HomHom: C(Y,Q)≤3 leaves only one significant count, C(X,P), meaning that both non-SNP and SNP positions were homozygous;

HetHom: X≠Y and P=Q, i.e. the non-SNP position was heterozygous and the SNP position was homozygous;

HomHet: X=Y and P≠Q, i.e. the non-SNP position was homozygous and the SNP position was heterozygous;

HetHet: X≠Y and P≠Q, i.e. both the non-SNP and SNP positions were heterozygous.

The HomHet and HetHet conditions with heterozygous SNP positions were used to distinguish read counts from cancer and non-cancer cells. For these conditions, the third maximum count of the matrix, C(Z,P) or C(Z,Q), can be attributed to a somatic mutation of a cancer cell.

The third maximum count can be used to detect a somatic mutation when the count is significantly above the background sequencing error rate. The average error rate, E, was calculated from all other counts, except for the highest three counts.

A Phred-like significance score for a somatic mutation, which is a Chi-squared probability with one degree of freedom, was calculated with Formula I:

S=(C(Z,P)²/(C(Z,P)+C(X,P))+(C(Z,P)−E)²/E)/2*10 Formula I

The TMB level is the number of positions having S>30, normalized by the total number of positions in the heterozygous SNP regions {N(HomHet)+N(HetHet)} in Mbases, as shown in Formula II:

TMB=N(S>30)/(N(HomHet)+N(HetHet))*1000000 Formula II

The median sequence length used to calculate TMB was 9.7 Mb for WES, 4.6 Mb for MYCHOICE HRD-PLUS with germline subtraction, and 1.9 Mb for the unique algorithm of this invention that did not require germline subtraction.

Results were compared for the three different methods for determining TMB. The comparison showed that the unique algorithm of this invention that does not require germline subtraction provided surprisingly accurate TMB values. The comparison of TMB results is shown in Table 4.

TABLE 4
Comparison of TMB levels obtained with and without germline subtraction

| | WES with | MYCHOICE HRD- | This invention |
|---|---|---|---|
| | germline | PLUS with germline | without germline |
| | subtraction | subtraction | subtraction |
| WES with germline | — | 1.6** | 1.5** |
| subtraction | | p = 4.6 × 10⁻⁶ | p = 1.2 × 10⁻⁵ |
| MYCHOICE HRD- | 0.895* | — | 0.04** |
| PLUS with germline | | | p = 0.88 |
| subtraction | | | |
| This invention without | 0.908* | 0.834* | — |
| germline subtraction | | | |

*Correlation coefficient.
**Mean difference in variants per Mb (with p value).

The correlation coefficients in Table 4 show that the method of this invention using a unique algorithm that does not require germline subtraction provided surprisingly accurate TMB values as compared to WES-based conventional methods with germline subtraction, as well as MYCHOICE HRD-PLUS with germline subtraction.

Thus, the method of this invention using a unique algorithm that does not require germline subtraction is unexpectedly advantageous because it does not require a germline comparator sample and can be performed on any sample containing cancer and non-cancer cells.

The method of this invention using a unique algorithm that does not require germline subtraction is a powerul tool because a threshold or reference for TMB level can be determined for each disease or population to be evaluated.

What is claimed is:

1. A method for detecting a somatic variant, comprising:
(a) sequencing cells of a sample;
(b) identifying a set of heterozygous SNP positions, wherein each SNP has alleles B and A;
(c) detecting two germline allele parings for a SNP position and a variant in a position near the SNP position, wherein the two germline allele parings are (i) allele B and a first variant allele, and (ii) allele A and a second variant allele which may the same or different than the first variant allele; and
(d) detecting a third allele pairing which is (iii) allele B and a third variant allele that is different from the first variant allele.

2. The method of claim 1, wherein the allele pairings are each detected in a contiguous nucleic acid sequence containing one of the SNP positions, so that the variant position is within one detection length of the SNP position.

3. The method of claim 2, wherein the contiguous nucleic acid sequence is a read length of about 100 to 5000 bases.

4. The method of claim 2, wherein the detection length is 200 to 1000 contiguous base positions on each flank of the SNP position.

5. The method of claim 1, wherein the method does not utilize a separate germline comparator sample.

6. The method of claim 1, wherein the sample is a cancer tissue sample, a sample of tumor cells, or a tumor sample.

7. The method of claim 1, wherein the amount of non-tumor cells in the sample is minimized.

8. The method of claim 1, wherein the tumor sample contains non-tumor cells.

9. The method of claim 1, wherein the allele pairings are detected by massively parallel sequencing, by hybridization, or with amplification.

10. The method of claim 1, wherein the set of heterozygous SNP positions is at least 5000 SNP positions, or at least 100,000 SNP positions, or at least 500,000 SNP positions, or at least 1,000,000 SNP positions, or at least 2,000,000 SNP positions.

11. The method of claim 1, wherein the method detects a somatic variant at a minimum level of 0.1 per Mb, or 0.3 per Mb, or 0.7 per Mb.

12. The method of claim 1, wherein the detecting is obtained with a targeted SNP panel.

13. The method of claim 1, wherein the detecting is obtained by fragmentation sequencing that uses a human reference genome.

14. A method for detecting a somatic variant, comprising:
(a) sequencing cells of a tumor sample;
(b) obtaining sequence reads from the sample using a massively parallel nucleic acid sequencing process, wherein the sequence reads have a read length;
(c) mapping the sequence reads to a reference genome;
(d) assembling a somatic variant count matrix of sequence reads that are mapped to a heterozygous-SNP position of the reference genome, wherein the count matrix has first and second elements which count allele pairings of SNP alleles B and A, respectively, to a variant allele, and wherein the count matrix has a third element which counts read sequences from SNP allele B paired to a different variant allele than in the first element; and
(e) calculating a somatic mutation significance score (S) for the third element.

15. The method of claim 14, wherein the method does not utilize a separate germline comparator sample.

16. The method of claim 14, wherein the sample is a cancer tissue sample, a sample of tumor cells, or a tumor sample.

17. The method of claim 14, wherein the method detects a somatic variant at a minimum level of 0.1 per Mb, or 0.3 per Mb, or 0.7 per Mb.

18. The method of claim 14, wherein the sequence reads are obtained with a targeted SNP panel.

19. The method of claim 14, wherein the read length is 100 to 5000, or 200 to 1000 contiguous base positions.

20. The method of claim 14, wherein the average read depth is at least 50x for the portion of the reference genome covered.

21. The method of claim 14, wherein the reference genome is a human genome.

22. The method of claim 14, wherein the sequence reads are error-filtered by one or more of the following steps:
ignoring reads with multiple map locations;
ignoring bases numbered 1-10 and greater than 86 in each read of length 100 bases;
matching map location size to insert size for forward and reverse reads of the same insert;
ignoring reads for which neither forward nor reverse reads overlap the SNP position; and
combining the base calls for forward and reverse reads which overlap, wherein the SNP calls are the same, and ignoring positions in the overlap with different base calls.

23. The method of claim 14, wherein the sequence reads are position-filtered by one or more of the following steps:
ignoring positions with ambiguous wild-type sequences;
ignoring positions with known SNP polymorphism;
ignoring positions with read depth less than 50;
ignoring repetitive positions for which an unrelated genomic segment was matched to the sequence; and
ignoring positions with unknown SNP polymorphism identified in a representative set of unrelated samples.

24. The method of claim 14, wherein the somatic mutation significance score (S) is given by Formula I
S=(C(Z,P)²/(C(Z,P)+C(X,P))+(C(Z,P)−E)²/E)/2*10 Formula I
wherein C(Z,P) is the third element count, C(X,P) is the first element count, and E is an error rate calculated from the average of all other counts in the matrix, except for the highest three counts, for all SNP regions.

25. A method for identifying a subject having cancer who benefits from a treatment, the method comprising:
(a) sequencing cells of a tumor sample from the subject;
(b) identifying a set of heterozygous SNP positions, wherein each SNP has alleles B and A;
(c) detecting two germline allele parings for a SNP position and a variant in a position near the SNP position, wherein the two germline allele parings are (i) allele B and a first variant allele, and (ii) allele A and a second variant allele which may the same or different than the first variant allele; and
(d) detecting a third allele pairing which is (iii) allele B and a third variant allele that is different from the first variant allele, wherein the third allele pairing arises from a somatic variant;
(f) calculating a value for a tumor mutation burden from the somatic variants detected from the allele pairings; and
(g) identifying the subject having cancer who benefits from a treatment who has the tumor mutation burden greater than a reference level.

26. A method for identifying a subject having cancer who benefits from a treatment, the method comprising:
(a) sequencing cells of a tumor sample from the subject;
(b) obtaining sequence reads from the sample using a massively parallel nucleic acid sequencing process, wherein the sequence reads have a read length;
(c) mapping the sequence reads to a reference genome;
(d) assembling a somatic variant count matrix of sequence reads that are mapped to a heterozygous-SNP position of the reference genome, wherein the count matrix has first and second elements which count allele pairings of SNP alleles B and A, respectively, to a variant allele, and wherein the count matrix has a third element which counts read sequences from SNP allele B paired to a different variant allele than in the first element;
(e) calculating a value for a tumor mutation burden of the sample by the steps:
(i) calculating a somatic mutation significance score (S) for the third element; and
(ii) calculating the value for the tumor mutation burden from the number of somatic variants having a somatic mutation significance score above a threshold, normalized by the total number of positions in the heterozygous-SNP regions; and
(f) identifying the subject having cancer who benefits from a treatment who has the tumor mutation burden greater than a reference level of somatic mutation.

27. The method of claim 26, wherein the number of heterozygous-SNPs in the reference genome is from about 100 up to the total number of heterozygous-SNPs in the reference genome.

28. The method of claim 25 or 26, wherein the reference level of somatic mutation is a level for which the subject will benefit from the treatment.

29. The method of claim 25 or 26, wherein the reference level of somatic mutation is the average tumor mutation burden of the reference genome.

30. The method of claim 25 or 26, wherein the reference level of somatic mutation is the average tumor mutation burden of a reference population having the same kind of cancer as the subject.

31. The method of claim 25 or 26, wherein the reference level of somatic mutation is the average tumor mutation burden of a reference population not having cancer.

32. The method of claim 25 or 26, wherein the reference level of somatic mutation is the average tumor mutation burden of a reference population that does not benefit from the treatment.

33. The method of claim 25 or 26, wherein the reference level of somatic mutation is obtained with a different sample from the subject.

34. The method of claim 26, wherein the somatic mutation significance score (S) is greater than 15, or 20, or 30, or 40, and is given by Formula I
S=(C(Z,P)²/(C(Z,P)+C(X,P))+(C(Z,P)−E)²/E)/2*10 Formula I
wherein C(Z,P) is the third element count, C(X,P) is the first element count, and E is an error rate calculated from the average of all other counts in the matrix, except for the highest three counts, for all SNP regions.

35. The method of claim 26, wherein the tumor mutation burden threshold is 15, or 20, or 30, or 40, and the tumor mutation burden is given by Formula II
TMB=N(S>threshold)/(N(HomHet)+N(HetHet))*1000000 Formula II
wherein N is the number of somatic variants having a somatic mutation significance score above the threshold, normalized by the total number of positions in the heterozygous-SNP regions (N(HomHet) +N(HetHet)).

36. A method for treating cancer in a subject in need thereof, the method comprising:
(a) sequencing cells of a tumor sample from the subject;
(b) identifying a set of heterozygous SNP positions, wherein each SNP has alleles B and A;
(c) detecting two germline allele parings for a SNP position and a variant in a position near the SNP position, wherein the two germline allele parings are (i) allele B and a first variant allele, and (ii) allele A and a second variant allele which may the same or different than the first variant allele; and
(d) detecting a third allele pairing which is (iii) allele B and a third variant allele that is different from the first variant allele, wherein the third allele pairing arises from a somatic variant;
(e) calculating a value for a tumor mutation burden from the somatic variants detected;
(f) identifying the subject having cancer who benefits from a treatment who has the tumor mutation burden greater than a reference level; and
(g) administering a treatment for cancer.

37. A method for treating cancer in a subject in need thereof, the method comprising:
(a) sequencing cells of a tumor sample from the subject;
(b) obtaining sequence reads from the sample using a massively parallel nucleic acid sequencing process, wherein the sequence reads have a read length;
(c) mapping the sequence reads to a reference genome;
(d) assembling a somatic variant count matrix of sequence reads that are mapped to a heterozygous-SNP position of the reference genome, wherein the count matrix has first and second elements which count allele pairings of SNP alleles B and A, respectively, to a variant allele, and wherein the count matrix has a third element which counts read sequences from SNP allele B paired to a different variant allele than in the first element;
(e) calculating a value for a tumor mutation burden of the sample by the steps:
(i) calculating a somatic mutation significance score (S) for the third element for each somatic variant; and
(ii) calculating the value for the tumor mutation burden from the number of somatic variants having a somatic mutation significance score above a threshold, normalized by the total number of positions in the heterozygous-SNP regions;
(f) identifying the subject having cancer who will benefit from a treatment who has the tumor mutation burden greater than a reference level of somatic mutation; and
(g) administering a treatment for cancer.

38. The method of claim 37, wherein the treatment for cancer comprises administering an immune checkpoint inhibitor drug.

39. The method of claim 36 or 37, wherein the reference level of somatic mutation is a level for which the subject will benefit from the treatment.

40. The method of claim 36 or 37, wherein the reference level of somatic mutation is the average tumor mutation burden of the reference genome.

41. The method of claim 36 or 37, wherein the reference level of somatic mutation is the average tumor mutation burden of a reference population having the same kind of cancer as the subject.

42. The method of claim 36 or 37, wherein the reference level of somatic mutation is the average tumor mutation burden of a reference population not having cancer.

43. The method of claim 36 or 37, wherein the reference level of somatic mutation is the average tumor mutation burden of a reference population that does not benefit from the treatment.

44. A method for treating cancer in a subject in need thereof, the method comprising:
(a) sequencing cells of a tumor sample from the subject;
(b) obtaining sequence reads from the sample using a massively parallel nucleic acid sequencing process, wherein the sequence reads have a read length;
(c) mapping the sequence reads to a reference genome;
(d) assembling a somatic variant count matrix of sequence reads that are mapped to a heterozygous-SNP position of the reference genome, wherein the count matrix has first and second elements which count allele pairings of SNP alleles B and A, respectively, to a variant allele, and wherein the count matrix has a third element which counts read sequences from SNP allele B paired to a different variant allele than in the first element;
(e) calculating a value for a tumor mutation burden of the sample by the steps:
(i) calculating a somatic mutation significance score (S) for the third element for each somatic variant; and
(ii) calculating the value for the tumor mutation burden from the number of somatic variants having a somatic mutation significance score above a threshold, normalized by the total number of positions in the heterozygous-SNP regions;
(f) identifying a subject having cancer who will benefit from a treatment who has the tumor mutation burden greater than a reference level of somatic mutation;
(g) monitoring the subject for the signs and symptoms of cancer for a period of time; and
(h) administering a treatment for cancer.

45. The method of claim 44, wherein the treatment is administering an immune checkpoint inhibitor.

46. The method of claim 44, wherein the reference level of somatic mutation is a level for which the subject will benefit from the treatment.

47. The method of claim 44, wherein the reference level of somatic mutation is the average tumor mutation burden of the reference genome.

48. The method of claim 44, wherein the reference level of somatic mutation is the average tumor mutation burden of a reference population having the same kind of cancer as the subject.

49. The method of claim 44, wherein the reference level of somatic mutation is the average tumor mutation burden of a reference population not having cancer.

50. The method of claim 44, wherein the reference level of somatic mutation is the average tumor mutation burden of a reference population that does not benefit from the treatment.

51. A method for monitoring a response of a subject having cancer to a treatment, the method comprising:
(a) sequencing cells of a tumor sample from the subject;
(b) identifying a set of heterozygous SNP positions, wherein each SNP has alleles B and A;
(c) detecting two germline allele parings for a SNP position and a variant in a position near the SNP position, wherein the two germline allele parings are (i) allele B and a first variant allele, and (ii) allele A and a second variant allele which may the same or different than the first variant allele; and
(d) detecting a third allele pairing which is (iii) allele B and a third variant allele that is different from the first variant allele, wherein the third allele pairing arises from a somatic variant;
(e) calculating a value for a tumor mutation burden from the somatic variants detected.

52. A method for monitoring a response of a subject having cancer to a treatment, the method comprising:
(a) sequencing cells of a tumor sample from the subject;
(b) obtaining sequence reads from the sample using a massively parallel nucleic acid sequencing process, wherein the sequence reads have a read length;
(c) mapping the sequence reads to a reference genome;
(d) assembling a somatic variant count matrix of sequence reads that are mapped to a heterozygous-SNP position of the reference genome, wherein the count matrix has first and second elements which count allele pairings of SNP alleles B and A, respectively, to a variant allele, and wherein the count matrix has a third element which counts read sequences from SNP allele B paired to a different variant allele than in the first element;
(e) calculating a value for a tumor mutation burden of the sample by the steps:
(i) calculating a somatic mutation significance score (S) for the third element for each somatic variant; and
(ii) calculating the value for the tumor mutation burden from the number of somatic variants having a somatic mutation significance score above a threshold, normalized by the total number of positions in the heterozygous-SNP regions.

53. A method for prognosing a subject having cancer, the method comprising:
(a) sequencing cells of a tumor sample from the subject;
(b) identifying a set of heterozygous SNP positions, wherein each SNP has alleles B and A;
(c) detecting two germline allele parings for a SNP position and a variant in a position near the SNP position, wherein the two germline allele parings are (i) allele B and a first variant allele, and (ii) allele A and a second variant allele which may the same or different than the first variant allele; and
(d) detecting a third allele pairing which is (iii) allele B and a third variant allele that is different from the first variant allele, wherein the third allele pairing arises from a somatic variant;
(e) calculating a value for a tumor mutation burden from the somatic variants detected; and
(f) prognosing the subject as having a poor prognosis who has the tumor mutation burden greater than a TMB reference level.

54. A method for prognosing a subject having cancer, the method comprising:
(a) sequencing cells of a tumor sample from the subject;
(b) obtaining sequence reads from the sample using a massively parallel nucleic acid sequencing process, wherein the sequence reads have a read length;
(c) mapping the sequence reads to a reference genome;
(d) assembling a somatic variant count matrix of sequence reads that are mapped to a heterozygous-SNP position of the reference genome, wherein the count matrix has first and second elements which count allele pairings of SNP alleles B and A, respectively, to a variant allele, and wherein the count matrix has a third element which counts read sequences from SNP allele B paired to a different variant allele than in the first element;
(e) calculating a value for a tumor mutation burden of the sample by the steps:
(i) calculating a somatic mutation significance score (S) for the third element for each somatic variant; and
(ii) calculating the value for the tumor mutation burden from the number of somatic variants having a somatic mutation significance score above a threshold, normalized by the total number of positions in the heterozygous-SNP regions;
(f) prognosing the subject as having a poor prognosis who has the tumor mutation burden greater than a TMB reference level; and
(g) administering a treatment for cancer.

55. The method of claim 54, wherein the treatment is administering an immune checkpoint inhibitor.

56. A kit for identifying a subject having cancer who benefits from a treatment, the kit comprising:
(a) reagents for obtaining sequence reads from a sample from the subject, wherein the sequence reads can be used to obtain a value for a tumor mutation burden of the sample; and
(b) instructions for using the reagents for obtaining the sequence reads and the value for a tumor mutation burden for identifying the subject.

57. A system for detecting a somatic variant, comprising:
means for receiving, enriching and amplifying a nucleic acid from a sample, wherein the sample contains cancer cells and non-cancer cells;
means for synthesizing a library from the nucleic acid;
means for contacting the library with a sequencing chip;
means for detecting a sequence in the library and transferring sequence data to a processor;
one or more processors for carrying out the steps:
(a) providing a sample which contains cancer cells and non-cancer cells;
(b) obtaining sequence reads from the sample using a massively parallel nucleic acid sequencing process, wherein the sequence reads have a read length;
(c) mapping the sequence reads to a reference genome;
(d) assembling a somatic variant count matrix of sequence reads that are mapped to a heterozygous-SNP position of the reference genome, wherein the count matrix has first and second elements which count allele pairings of SNP alleles B and A, respectively, to a variant allele, and wherein the count matrix has a third element which counts read sequences from SNP allele B paired to a different variant allele than in the first element;
(e) calculating a value for a tumor mutation burden of the sample by the steps:
(i) calculating a somatic mutation significance score (S) for the third element for each somatic variant; and
(ii) calculating the value for the tumor mutation burden from the number of somatic variants having a somatic mutation significance score above a threshold, normalized by the total number of positions in the heterozygous-SNP regions; and
a display for displaying, charting and reporting sequence information.

58. A non-transitory machine-readable storage medium having stored therein instructions for execution by a processor which cause the processor to perform the steps of a method for detecting a somatic variant, the method comprising:
(a) providing a sample which contains cancer cells and non-cancer cells;
(b) obtaining sequence reads from the sample using a massively parallel nucleic acid sequencing process, wherein the sequence reads have a read length;
(c) mapping the sequence reads to a reference genome;
(d) assembling a somatic variant count matrix of sequence reads that are mapped to a heterozygous-SNP position of the reference genome, wherein the count matrix has first and second elements which count allele pairings of SNP alleles B and A, respectively, to a variant allele, and wherein the count matrix has a third element which counts read sequences from SNP allele B paired to a different variant allele than in the first element;
(e) calculating a value for a tumor mutation burden of the sample by the steps:
(i) calculating a somatic mutation significance score (S) for the third element for each somatic variant; and
(ii) calculating the value for the tumor mutation burden from the number of somatic variants having a somatic mutation significance score above a threshold, normalized by the total number of positions in the heterozygous-SNP regions; and
(f) displaying, charting and reporting sequence information from the sample.
